(19) United States
(12) Patent Application Publication
(10) Pub. No.: US 2009/0181975 A1
(43) Pub. Date: Jul. 16, 2009

(54) NEBIVOLOL IN THE TREATMENT OF SEXUAL DYSFUNCTION

(75) Inventors: Inigo Saenz de Tejada, Madrid (ES); Javier Angulo, Madrid (ES); Sandeep Gupta, Plainsboro, NJ (US)

Correspondence Address: Forest Laboratories, Inc.;Attn: Charles S. Ryan, 909 3rd Avenue, New York, NY, 10022, US

(73) Assignee: FOREST LABORATORIES HOLDINGS LIMITED, Hamilton (BM)

(21) Appl. No.: 12/353,443

(22) Filed: Jan. 14, 2009

Related U.S. Application Data
(60) Provisional application No. 61/021,062, filed on Jan. 15, 2008.

(51) Int. Cl. A61K 31/4985; A61K 31/352; A61P 15/12; A61P 15/10; A61K 31/519

(52) U.S. Cl. 514/250; 514/456; 514/262.1

(57) ABSTRACT

The present invention provides methods of treating sexual dysfunction. The methods include administering an effective amount of nebivolol, or a pharmaceutically acceptable salt thereof, alone or in combination with a second active agent e.g. a PDE-5 inhibitor, such as sildenafil citrate. The methods of the present invention are particularly suited to the treatment of erectile dysfunction and female sexual arousal disorder.

This application claims the benefit of U.S. Provisional Application Ser. No. 61/021,062, filed Jan. 15, 2008, the entire disclosure of which is hereby incorporated by reference.

### FIELD OF THE INVENTION

The present invention relates to methods of treating sexual dysfunction. The methods include administering a therapeutically effective amount of nebivolol, or a pharmaceutically acceptable salt thereof, alone or in combination with a second active agent, e.g., a phosphodiesterase type V (PDE-5) inhibitor. Some methods of the present invention relate to the treatment of erectile dysfunction and female sexual arousal disorder.

### BACKGROUND OF THE INVENTION

Nebivolol is a next generation cardioselective beta blocker that promotes vasodilation through a nitric oxide (NO)-dependent mechanism.

α,α′-Iminobis(methylene)bis[2-chromanmethanol] derivatives are disclosed, for example, in EP-0,145,067 as β-1 blocking agents having therapeutic potential for treating hypertension. Nebivolol, which comprises a racemic mixture of (RSSS) and (SRRR) α,α′-iminobis(methylene)bis[6-fluoro-2-chromanmethanol] is disclosed, for example, in EP-0,145,067. Nebivolol is disclosed, for example, in EP-0,334,429.

The (SRRR) enantiomer has been shown to be a potent and selective β-1 blocking agent. The (RSSS) enantiomer has been shown not to act as a potent β-1 blocking agent, but rather as a potentiator for a series of antihypertensive agents such as atenolol, propranolol, prazosin, hydralazine and, interestingly, also its own (SRRR) enantiomer. The (RSSS) enantiomer has also been shown to contribute to several beneficial hemodynamic effects of nebivolol that distinguish it from other β-1 blocking agents, e.g., it acutely lowers blood pressure in spontaneous hypertensive rats, decreases total peripheral vascular resistance, and augments stroke volume in anaesthetized dogs.

Sexual dysfunction implies an inability to fully enjoy sexual intercourse. Sexual dysfunction includes disorders that interfere with a full sexual response cycle. Sexual dysfunction can affect mental health by inducing depression, anxiety, and debilitating feelings of inadequacy in affected individuals. Sexual dysfunction could be present throughout life. Alternatively, or in addition, sexual dysfunction can be acquired, situational, and/or generalized.

Erectile dysfunction is the persistent inability to obtain or maintain penile erection sufficient for satisfactory sexual performance. A number of type V phosphodiesterase (PDE-5) inhibitors have been useful in the treatment of erectile dysfunction. Sildenafil citrate is the first PDE-5 inhibitor to be approved for the treatment of erectile dysfunction. Other PDE-5 inhibitors useful in the treatment of ED include, e.g., tadalafil, vardenafil and zaprinast. ED is common in patients with hypertension. β-adrenergic receptor antagonists (β-ARAs) or beta blockers are some of the most widely prescribed treatments for hypertension. Sexual dysfunction, in particular, erectile dysfunction is reported to be a major side effect in hypertensive patients being treated with beta blockers. Thus, there is a need in the art for new methods of treating sexual dysfunction.

The following references may provide relevant background to the present invention: Doumas et al., Asian J. Androl., 2006, 8(2): 177-182; Rosenkranz et al., Life Sciences, 2006, 1103-1107).

### SUMMARY OF THE INVENTION

The present invention provides methods of treating sexual dysfunction by administering a therapeutically effective amount of nebivolol, or a pharmaceutically acceptable salt thereof. In certain embodiments, human penile resistance arteries are relaxed by at least about 2.5%

In addition, the present invention provides methods of enhancing PDE-5 inhibitor-induced relaxation of human corpus cavernosum tissue in a patient receiving a PDE-5 inhibitor, by administering a therapeutically effective amount of nebivolol, or a pharmaceutically acceptable salt thereof. In certain embodiments, PDE-5 inhibitor-induced relaxation of human corpus cavernosum tissue is enhanced by at least 2.5%, as compared to treatment with a PDE-5 inhibitor alone.

In addition, the present invention provides methods of enhancing PDE-5 inhibitor-mediated dilation of human penile resistance arteries in a patient by administering a therapeutically effective amount of nebivolol, or a pharmaceutically acceptable salt thereof. In certain embodiments, PDE-5 inhibitor-mediated dilation of human penile resistance arteries is enhanced by at least 2.5%, as compared to treatment with a PDE-5 inhibitor alone.

In addition, the present invention provides methods of treating sexual dysfunction by administering nebivolol, or a pharmaceutically acceptable salt thereof, in combination with a second active agent. According to some embodiments, the present invention provides methods of treating sexual dysfunction by administering a therapeutically effective amount of nebivolol, or a pharmaceutically acceptable salt thereof, in combination with a PDE-5 inhibitor, such as sildenafil citrate. In certain embodiments, relaxation of human corpus cavernosum tissue is enhanced by at least 2.5%, as compared to treatment with a PDE-5 inhibitor alone

In addition, the present invention provides methods of enhancing PDE-5 inhibitor-induced relaxation of human corpus cavernosum tissue in a patient receiving a PDE-5 inhibitor, by administering a therapeutically effective amount of nebivolol, or a pharmaceutically acceptable salt thereof, and a PDE-5 inhibitor. In certain embodiments, PDE-5 inhibitor-induced relaxation of human corpus cavernosum tissue is enhanced by at least 2.5%, as compared to treatment with a PDE-5 inhibitor alone.

In addition, the present invention provides methods of enhancing PDE-5 inhibitor-mediated dilation of human penile resistance arteries in a patient by administering a therapeutically effective amount of nebivolol, or a pharmaceutically acceptable salt thereof, and a PDE-5 inhibitor. In certain embodiments, PDE-5 inhibitor-mediated dilation of human penile resistance arteries is enhanced by at least 2.5%, as compared to treatment with a PDE-5 inhibitor alone.

In certain embodiments, the PDE-5 inhibitor is sildenafil, or a pharmaceutically acceptable salt thereof, e.g., sildenafil citrate.

In further embodiments, the sexual dysfunction is erectile dysfunction or female sexual arousal disorder.

### BRIEF DESCRIPTION OF THE DRAWINGS

FIG. 1 shows the effects of intravenous administration of nebivolol (3 mg/kg) or the vehicle (50% glycofurol) on mean arterial pressure (MAP) in anesthetized rats. Data are expressed as mean±SEM of MAP in mm Hg (A) and as mean±SEM of the percentage of MAP value determined before nebivolol or vehicle administration (B). n indicates the number of animals used for the experiments. ** indicates p<0.01, *** p<0.001 vs vehicle by unpaired t test.

FIG. 2 shows the effects of intravenous administration of nebivolol (3 mg/kg) or the vehicle (50% glycofurol) on heart rate (HR) in anesthetized rats. Data are expressed as mean±SEM of HR in beats per minute (bpm) (A) and as mean±SEM of the percentage of HR value determined before nebivolol or vehicle administration (B). n indicates the number of animals used for the experiments. * indicates p<0.05, ** p<0.01 vs vehicle by unpaired t test.

FIG. 3 shows the effects of the nitric oxide synthase inhibitor, N_{ω}-nitro-L-arginine methyl ester (L-NAME, 3 mg/kg, i.v.) on hypotension caused by intravenous administration of nebivolol (3 mg/kg) in anesthetized rats. Data are expressed as mean±SEM of mean arterial pressure (MAP) in mm Hg (A) and as mean±SEM of the percentage of MAP value determined before nebivolol administration (B). n indicates the number of animals used for the experiments. * indicates p<0.05, ** p<0.01 vs control by unpaired t test.

FIG. 4 shows the effects of the nitric oxide synthase inhibitor, Nω-nitro-L-arginine methyl ester (L-NAME, 3 mg/kg, i.v.) on heart rate (HR) reduction caused by intravenous administration of nebivolol (3 mg/kg) in anesthetized rats. Data are expressed as mean±SEM of HR in beats per minute (bpm) (A) and as mean±SEM of the percentage of HR value determined before nebivolol administration (B). n indicates the number of animals used for the experiments.

FIG. 5 shows the effects of the guanylyl cyclase inhibitor, ODQ (1 mg/kg, i.v.), on hypotension caused by intravenous administration of nebivolol (3 mg/kg) in anesthetized rats. Data are expressed as mean±SEM of mean arterial pressure (MAP) in mm Hg (A) and as mean±SEM of the percentage of MAP value determined before nebivolol administration (B). n indicates the number of animals used for the experiments.

FIG. 6 shows the effects of the guanylyl cyclase inhibitor, ODQ (1 mg/kg, i.v.), on heart rate (HR) reduction caused by intravenous administration of nebivolol (3 mg/kg) in anesthetized rats. Data are expressed as mean±SEM of HR in beats per minute (bpm) (A) and as mean±SEM of the percentage of HR value determined before nebivolol administration (B). n indicates the number of animals used for the experiments.

FIG. 7 shows the effects of the β-adrenergic receptor blocker, propranolol (1 mg/kg, i.v.), on hypotension caused by intravenous administration of nebivolol (3 mg/kg) in anesthetized rats. Data are expressed as mean±SEM of mean arterial pressure (MAP) in mm Hg (A) and as mean±SEM of the percentage of MAP value determined before nebivolol administration (B). n indicates the number of animals used for the experiments.

FIG. 8 shows the effects of the β-adrenergic receptor blocker, propranolol (1 mg/kg, i.v.), on heart rate (HR) reduction caused by intravenous administration of nebivolol (3 mg/kg) in anesthetized rats. Data are expressed as mean±SEM of HR in beats per minute (bpm) (A) and as mean±SEM of the percentage of HR value determined before nebivolol administration (B). n indicates the number of animals used for the experiments. * indicates p<0.05, ** p<0.01 vs control by unpaired t test.

FIG. 9 shows the effects of the type 5 phosphodiesterase (PDE5) inhibitor, sildenafil (0.3 mg/kg, i.v.), on hypotension caused by intravenous administration of nebivolol (1 mg/kg) in anesthetized rats. Data are expressed as mean±SEM of mean arterial pressure (MAP) in mm Hg (A) and as mean±SEM of the percentage of MAP value determined before nebivolol administration (B). n indicates the number of animals used for the experiments. * indicates p<0.05 vs control by unpaired t test.

FIG. 10 shows the effects of the type 5 phosphodiesterase (PDE5) inhibitor, sildenafil (0.3 mg/kg, i.v.), on heart rate (HR) reduction caused by intravenous administration of nebivolol (1 mg/kg) in anesthetized rats. Data are expressed as mean±SEM of HR in beats per minute (bpm) (A) and as mean±SEM of the percentage of HR value determined before nebivolol administration (B). n indicates the number of animals used for the experiments. * indicates p<0.05, ** p<0.01 vs control by unpaired t test.

FIG. 11 shows the effects of the type 5 phosphodiesterase (PDE5) inhibitor, sildenafil (0.3 mg/kg, i.v.), on hypotension caused by intravenous administration of nebivolol (3 mg/kg) in anesthetized rats. Data are expressed as mean±SEM of mean arterial pressure (MAP) in mm Hg (A) and as mean±SEM of the percentage of MAP value determined before nebivolol administration (B). n indicates the number of animals used for the experiments.

FIG. 12 shows the effects of the type 5 phosphodiesterase (PDE5) inhibitor, sildenafil (0.3 mg/kg, i.v.), on heart rate (HR) reduction caused by intravenous administration of nebivolol (3 mg/kg) in anesthetized rats. Data are expressed as mean±SEM of HR in beats per minute (bpm) (A) and as mean±SEM of the percentage of HR value determined before nebivolol administration (B). n indicates the number of animals used for the experiments.

FIG. 13 shows the effects of intravenous administration of nebivololol (3 mg/kg; i.v.), sildenafil (0.3 mg/kg; i.v.) or the vehicle (50% glycofurol) on nitrite plus nitrate (NOx) concentration in rat serum and influence of Nω-nitro-L-arginine methyl ester (L-NAME, 3 mg/kg, i.v.), ODQ (1 mg/kg, i.v.), propranolol (1 mg/kg, i.v.) and sildenafil (0.3 mg/kg, i.v.) on nebivolol-induced increment in serum NOx concentration. Data are expressed as mean±SEM of the serum concentration of NOx (in μM). Numbers of animals used for determinations are in parenthesis. * indicates p<0.05 vs vehicle by unpaired t test.

FIG. 14 shows the effects of intravenous administration of atenolol (3 mg/kg), nebivolol (1 and 3 mg/kg; i.v.)(0.3 mg/kg; i.v.) or the vehicle (50% glycofurol) on nitrite plus nitrate (NOx) concentration in rat serum and influence of Nω-nitro-L-arginine methyl ester (L-NAME, 3 mg/kg, i.v.) on nebivolol-induced increment in serum NOx concentration. Serum concentrations of NOx are determined 10 min after nebivolol administration. Data are expressed as mean±SEM of the serum concentration of NOx (in μM). numbers of animals used for determinations are in parenthesis. ** p<0.01 vs vehicle and † p<0.05 vs nebivolol by one-factor ANOVA followed by Student-Newmann-Keuls test.

FIG. 15 shows the effects of intravenous administration of nebivololol (3 mg/kg; i.v.), sildenafil (0.3 mg/kg; i.v.) or the vehicle (50% glycofurol) on cyclic GMP (cGMP) concentration in rat plasma and influence of Nω-nitro-L-arginine methyl ester (L-NAME, 3 mg/kg, i.v.), ODQ (1 mg/kg, i.v.), propranolol (1 mg/kg, i.v.) and sildenafil (0.3 mg/kg, i.v.) on nebivolol-induced effects in plasma cGMP levels. Data are expressed as mean±SEM of the percentage of cGMP concentration determined before nebivolol administration in each rat. numbers of animals used for determinations are in parenthesis. ** indicates p<0.01 vs vehicle and † p<0.05 vs nebivolol and vs sildenafil by unpaired t test.

FIG. 16 shows the effects of intravenous administration of atenolol (3 mg/kg) and nebivolol (1 and 3 mg/kg) on cyclic GMP (cGMP) concentration in rat plasma cGMP concentration. Plasma concentrations of GMP are determined 10 min after nebivolol administration. Data are expressed as mean±SEM of the cGMP concentration in pmol/mL in A, while in B, data are expressed as mean±SEM of the percentage of cGMP concentration determined before vehicle (50% glycofurol) or drug administration in each rat. numbers of animals used for determinations are in parenthesis. *** p<0.001 vs basal (A) or vehicle (B), † p<0.05 vs 3 mg/kg nebivolol by one-factor ANOVA followed by Student-Newmann-Keuls test.

FIG. 17 shows the influence of intravenous administration of nebivolol (1 and 3 mg/kg) on the effects of sildenafil (0.3 mg/kg; i.v.) on cyclic GMP (cGMP) concentration in rat plasma. Plasma concentrations of GMP are determined 10 min after nebivolol administration. Data are expressed as mean±SEM of the cGMP concentration in pmol/mL. Numbers of animals used for determinations are in parenthesis. * p<0.05, ** p<0.01, *** p<0.001 vs basal, † p<0.05, ††† p<0.001 vs sildenafil+nebivolol 3 mg/kg by one-factor ANOVA followed by Student-Newmann-Keuls test.

FIG. 18 shows a comparison of the effects of intravenous administration of nebivolol (3 mg/kg) and sodium nitroprusside (SNP, 0.1 mg/kg) on cyclic GMP (cGMP) concentration in rat plasma cGMP concentration. Plasma concentrations of GMP are determined 10 min after nebivolol administration. Data are expressed as mean±SEM of the cGMP concentration in pmol/mL in A, while in B, data are expressed as mean±SEM of the percentage of cGMP concentration determined before vehicle (50% glycofurol) or drug administration in each rat. numbers of animals used for determinations are in parenthesis. * p<0.05, ** p<0.01, *** p<0.001 vs basal (A) or vehicle (B) by one-factor ANOVA followed by Student-Newmann-Keuls test.

FIG. 19 shows the effects of intravenous administration of vehicle (50% glycofurol) on erectile responses induced by cavernosal nerve electrical stimulation (CNES) in anesthetized rats. Data are expressed as mean±SEM of the area under the curve (AUC) of the intracavernosal pressure (ICP) increase to CNES normalized by mean arterial pressure value at each stimulation. n indicates the number of animals used for the experiments.

FIG. 20 shows the effects of intravenous administration of nebivolol (3 mg/kg) on erectile responses induced by cavernosal nerve electrical stimulation (CNES) in anesthetized rats. Data are expressed as mean±SEM of the area under the curve (AUC) of the intracavernosal pressure (ICP) increase to CNES normalized by mean arterial pressure value at each stimulation. n indicates the number of animals used for the experiments. * indicates p<0.05, ** p<0.01 vs control by one-ANOVA followed by Student-Newmann-Keuls test.

FIG. 21 shows the effects of intravenous administration of nebivolol (3 mg/kg) on erectile responses induced by cavernosal nerve electrical stimulation (CNES) in anesthetized rats. Data are expressed as mean±SEM of the area under the curve (AUC) of the intracavernosal pressure (ICP) increase to CNES. n indicates the number of animals used for the experiments. ** indicates p<0.01 vs control by one-ANOVA followed by Student-Newmann-Keuls test.

FIG. 22 shows the relaxant responses induced by nebivolol (1 nM to 0.1 mM) on human corpus cavernosum strips contracted with the thromboxane receptor agonist, U46619 (1-3 nM). Data are expressed as mean±SEM of the percentage of maximum relaxation. n indicates number of patients from whom the tissues were collected for the study.

FIG. 23 shows the effects of the vehicle (0.01% DMSO) (A) or nebivolol (0.3 μM) (B) on endothelium-dependent relaxation to acetylcholine (ACh; 1 nM to 0.1 mM) in human corpus cavernosum strips contracted with the thromboxane analogue, U46619 (1-3 nM). Data are expressed as mean±SEM of the percentage of maximum relaxation. n indicates number of patients from whom the tissues were collected for the study.

FIG. 24 shows the effects of atenolol (1 μM) (A) or metoprolol (1 μM) (B) on endothelium-dependent relaxation to acetylcholine (ACh; 1 nM to 0.1 mM) in human corpus cavernosum strips contracted with the thromboxane analogue, U46619 (1-3 nM). Data are expressed as mean±SEM of the percentage of maximum relaxation. n indicates number of patients from whom the tissues were collected for the study.

FIG. 25 shows the effects of the vehicle (0.01%) on endothelium-dependent relaxation to acetylcholine (ACh; 1 nM to 0.1 mM) in human corpus cavernosum strips contracted with the thromboxane receptor agonist, U46619 (1-3 nM). Data are expressed as mean±SEM of the percentage of maximum relaxation. n indicates number of patients from whom the tissues were collected for the study.

FIG. 26 shows the effects of the nebivolol (10 μM) on endothelium-dependent relaxation to acetylcholine (ACh; 1 nM to 0.1 mM) in human corpus cavernosum strips contracted with the thromboxane receptor agonist, U46619 (1-3 nM). Data are expressed as mean±SEM of the percentage of maximum relaxation. n indicates number of patients from whom the tissues were collected for the study. * indicates p<0.05 vs control by two-factors ANOVA.

FIG. 27 shows the effects of the nebivolol (10 μM) on relaxation induced by the PDE5 inhibitor, sildenafil (1 nM to 10 μM) in human corpus cavernosum strips contracted with the thromboxane receptor agonist, U46619 (1-3 nM). Data are expressed as mean±SEM of the percentage of maximum relaxation. n indicates number of patients from whom the tissues were collected for the study.

FIG. 28 shows the effects of nebivolol (10 μM (A) and 1 μM (B) on relaxation induced by the PDE5 inhibitor, sildenafil (1 nM to 10 μM) in human corpus cavernosum strips contracted with the thromboxane analogue, U46619 (1-3 nM). Data are expressed as mean±SEM of the percentage of maximum relaxation. n indicates number of patients from whom the tissues were collected for the study. *** p<0.001 vs control by two-factors ANOVA.

FIG. 29 shows the effects of atenolol (1 μM) on relaxation induced by the PDE5 inhibitor, sildenafil (1 nM to 10 μM) in human corpus cavernosum strips contracted with the thromboxane analogue, U46619 (1-3 nM). Data are expressed as mean±SEM of the percentage of maximum relaxation. n indicates number of patients from whom the tissues were collected for the study.

FIG. 30 shows the influence of the endothelium on relaxant responses induced by nebivolol (1 nM to 0.1 mM) in human penile resistance arteries contracted with the thromboxane receptor agonist, U46619 (30-100 nM). Data are expressed as mean±SEM of the percentage of maximum relaxation. n indicates number of patients from whom the tissues were collected for the study.

FIG. 31 shows the relaxant responses induced by metoprolol, atenolol, nebivolol (1 nM to 0.1 mM) or the vehicle (0.01% DMSO) in intact human penile resistance arteries (HPRA) contracted with the thromboxane receptor agonist, U46619 (30-100 nM). The effect of nebivolol on denuded HPRA is also shown (nebivolol-E). Data are expressed as mean±SEM of the percentage of maximum relaxation. n indicates number of patients from whom the tissues were collected for the study. *** p<0.001 vs metoprolol, atenolol or vehicle by a two-factors ANOVA.

FIG. 32 shows the effects of metoprolol (1 μM) (A) or atenolol (1 μM) (B) on endothelium-dependent relaxation to acetylcholine (ACh; 1 nM to 10 μM) in human penile resistance arteries contracted with the thromboxane analogue, U46619 (30-100 nM). Data are expressed as mean±SEM of the percentage of maximum relaxation. n indicates number of patients from whom the tissues were collected for the study.

FIG. 33 shows the effects of nebivolol (0.3 μM) (B) on endothelium-dependent relaxation to acetylcholine (ACh; 1 nM to 10 μM) in human penile resistance arteries contracted with the thromboxane analogue, U46619 (30-100 nM). Data are expressed as mean±SEM of the percentage of maximum relaxation. n indicates number of patients from whom the tissues were collected for the study.

FIG. 34 shows the effects of (A) atenolol 1 μM and (B) nebivolol 1 μM on relaxations induced by the PDE5 inhibitor sildenafil (1 nM to 10 μM) in intact human penile resistance arteries (HPRA) contracted with the thromboxane receptor agonist U46619 (10 nM to 30 nM). Data are expressed as mean±SEM of the percentage of maximum relaxation induced by papaverine 0.1 mM at the end of the experiment. n indicates the number of patients from whom the tissues were collected for the experiments. ***P<0.001 vs vehicle by a two-factors ANOVA test.

FIG. 35 shows glycemia levels before (white bars) and after (dark bars) treating the rats for 10 days with vehicle (DMSO/PEG 300, 1:1; 12 μL/day s.c.), atenolol or nebivolol (both at 3 and 10 mg/kg/day s.c. for 10 days in DMSO/PEG 300, 1:1 at 12 μL/day rate) after 8 weeks of untreated diabetes. Data are expressed as the mean±SEM of the blood glucose concentration values (in mg/dL). The number of animals used is in parenthesis.

FIG. 36 shows mean arterial pressure (MAP) (A) and heart rate (HR) (B) values in non diabetic rats and in diabetic rats treated for 10 days with vehicle (DMSO/PEG 300, 1:1; 12 μL/day s.c.), atenolol or nebivolol (both at 3 and 10 mg/kg/day s.c. for 10 days in DMSO/PEG 300, 1:1 at 12 μL/day rate) after 8 weeks of untreated diabetes. Data are expressed as the mean±SEM. n indicates the number of animals used. ** indicates p<0.01, *** p<0.001 vs No diabetes and † p<0.05, †† p<0.01 vs vehicle by a one-factor ANOVA test followed by a Student-Newmann-Keuls test

FIG. 37 shows the effects of intravenous administration of sildenafil (0.3 mg/kg) on erectile responses elicited by cavernosal nerve electrical stimulation in anesthetized diabetic rats treated with vehicle (DMSO/PEG 300, 1:1; 12 μL/day s.c. for 10 days) after 8 weeks of untreated diabetes. Data are expressed as the mean±SEM of the area under the curve (AUC) of intracavernosal pressure (ICP) increase to cavernosal nerve stimulation (in mm Hg×s) normalized by mean arterial pressure (MAP) values. n indicates the number of animals used.

FIG. 38 shows the effects of intravenous administration of sildenafil (0.3 mg/kg) on erectile responses elicited by cavernosal nerve electrical stimulation in anesthetized diabetic rats treated with vehicle (DMSO/PEG 300, 1:1; 12 μL/day s.c. for 10 days) after 8 weeks of untreated diabetes. Data are expressed as the mean±SEM of the intracavernosal pressure (ICP) increase to cavernosal nerve stimulation (in mm Hg) normalized by mean arterial pressure (MAP) values. n indicates the number of animals used.

FIG. 39 shows the effects of intravenous administration of sildenafil (0.3 mg/kg) on erectile responses elicited by cavernosal nerve electrical stimulation in anesthetized diabetic rats treated with atenolol (3 mg/kg/day s.c. for 10 days in DMSO/PEG 300, 1:1 at 12 μL/day rate) after 8 weeks of untreated diabetes. Data are expressed as the mean±SEM of the area under the curve (AUC) of intracavernosal pressure (ICP) increase to cavernosal nerve stimulation (in mm Hg×s) normalized by mean arterial pressure (MAP) values. n indicates the number of animals used.

FIG. 40 shows the effects of intravenous administration of sildenafil (0.3 mg/kg) on erectile responses elicited by cavernosal nerve electrical stimulation in anesthetized diabetic rats treated with atenolol (3 mg/kg/day s.c. for 10 days in DMSO/PEG 300, 1:1 at 12 μL/day rate) after 8 weeks of untreated diabetes. Data are expressed as the mean±SEM of the intracavernosal pressure (ICP) increase to cavernosal nerve stimulation (in mm Hg) normalized by mean arterial pressure (MAP) values. n indicates the number of animals used.

FIG. 41 shows the effects of intravenous administration of sildenafil (0.3 mg/kg) on erectile responses elicited by cavernosal nerve electrical stimulation in anesthetized diabetic rats treated with atenolol (10 mg/kg/day s.c. for 10 days in DMSO/PEG 300, 1:1 at 12 μL/day rate) after 8 weeks of untreated diabetes. Data are expressed as the mean±SEM of the area under the curve (AUC) of intracavernosal pressure (ICP) increase to cavernosal nerve stimulation (in mm Hg×s) normalized by mean arterial pressure (MAP) values. n indicates the number of animals used. * p<0.05 vs control by two-factors ANOVA test.

FIG. 42 shows the effects of intravenous administration of sildenafil (0.3 mg/kg) on erectile responses elicited by cavernosal nerve electrical stimulation in anesthetized diabetic rats treated with atenolol (10 mg/kg/day s.c. for 10 days in DMSO/PEG 300, 1:1 at 12 μL/day rate) after 8 weeks of untreated diabetes. Data are expressed as the mean±SEM of the intracavernosal pressure (ICP) increase to cavernosal nerve stimulation (in mm Hg) normalized by mean arterial pressure (MAP) values. n indicates the number of animals used.

FIG. 43 shows the effects of intravenous administration of sildenafil (0.3 mg/kg) on erectile responses elicited by cavernosal nerve electrical stimulation in anesthetized diabetic rats treated with nebivolol (3 mg/kg/day s.c. for 10 days in DMSO/PEG 300, 1:1 at 12 μL/day rate) after 8 weeks of untreated diabetes. Data are expressed as the mean±SEM of the area under the curve (AUC) of intracavernosal pressure (ICP) increase to cavernosal nerve stimulation (in mm Hg×s) normalized by mean arterial pressure (MAP) values. n indicates the number of animals used.

FIG. 44 shows the effects of intravenous administration of sildenafil (0.3 mg/kg) on erectile responses elicited by cavernosal nerve electrical stimulation in anesthetized diabetic rats treated with nebivolol (3 mg/kg/day s.c. for 10 days in DMSO/PEG 300, 1:1 at 12 μL/day rate) after 8 weeks of untreated diabetes. Data are expressed as the mean±SEM of the intracavernosal pressure (ICP) increase to cavernosal nerve stimulation (in mm Hg) normalized by mean arterial pressure (MAP) values. n indicates the number of animals used.

FIG. 45 shows the effects of intravenous administration of sildenafil (0.3 mg/kg) on erectile responses elicited by cavernosal nerve electrical stimulation in anesthetized diabetic rats treated with nebivolol (10 mg/kg/day s.c. for 10 days in DMSO/PEG 300, 1:1 at 12 μL/day rate) after 8 weeks of untreated diabetes. Data are expressed as the mean±SEM of the area under the curve (AUC) of intracavernosal pressure (ICP) increase to cavernosal nerve stimulation (in mm Hg×s) normalized by mean arterial pressure (MAP) values. n indicates the number of animals used.

FIG. 46 shows the effects of intravenous administration of sildenafil (0.3 mg/kg) on erectile responses elicited by cavernosal nerve electrical stimulation in anesthetized diabetic rats treated with nebivolol (10 mg/kg/day s.c. for 10 days in DMSO/PEG 300, 1:1 at 12 μL/day rate) after 8 weeks of untreated diabetes. Data are expressed as the mean±SEM of the intracavernosal pressure (ICP) increase to cavernosal nerve stimulation (in mm Hg) normalized by mean arterial pressure (MAP) values. n indicates the number of animals used.

FIG. 47 shows erectile responses elicited by cavernosal nerve electrical stimulation in anesthetized diabetic rats treated for 10 days with vehicle (DMSO/PEG 300, 1:1; 12 μL/day s.c.), atenolol or nebivolol (both at 3 and 10 mg/kg/day s.c. for 10 days in DMSO/PEG 300, 1:1 at 12 μL/day rate) after 8 weeks of untreated diabetes. Data are expressed as the mean±SEM of the area under the curve (AUC) of intracavernosal pressure (ICP) increase to cavernosal nerve stimulation (in mm Hg×s) normalized by mean arterial pressure (MAP) values. n indicates the number of animals used. *** p<0.001 vs No Diabetes, †† p<0.01 vs Diabetes and p<0.01 vs Diabetes+atenolol by a two-factors ANOVA test

FIG. 48 shows erectile responses elicited by cavernosal nerve electrical stimulation in anesthetized diabetic rats treated for 10 days with vehicle (DMSO/PEG 300, 1:1; 12 μL/day s.c.), atenolol or nebivolol (both at 3 and 10 mg/kg/day s.c. for 10 days in DMSO/PEG 300, 1:1 at 12 μL/day rate) after 8 weeks of untreated diabetes. Data are expressed as the mean±SEM of the intracavernosal pressure (ICP) increase to cavernosal nerve stimulation (in mm Hg) normalized by mean arterial pressure (MAP) values. n indicates the number of animals used. * p<0.05, ** p<0.01, *** p<0.001 vs No diabetes, † p<0.05, †† p <0.01 vs Diabetes and p<0.01 vs diabetes+atenolol by a two-factors ANOVA test.

FIG. 49 shows erectile responses elicited by cavernosal nerve electrical stimulation in anesthetized diabetic rats after intravenous administration of sildenafil (0.3 mg/kg) and previously treated for 10 days with vehicle (DMSO/PEG 300, 1:1; 12 μL/day s.c.), atenolol or nebivolol (both at 3 and 10 mg/kg/day s.c. for 10 days in DMSO/PEG 300, 1:1 at 12 μL/day rate) after 8 weeks of untreated diabetes. Data are expressed as the mean±SEM of the area under the curve (AUC) of intracavernosal pressure (ICP) increase to cavernosal nerve stimulation (in mm Hg×s) normalized by mean arterial pressure (MAP) values. n indicates the number of animals used. * p<0.05, ** p<0.01 vs No Diabetes, # p<0.05, § p<0.001 vs No Diabetes+sildenafil, † p<0.05, †† p<0.01 vs Diabetes and vs Diabetes+atenolol by a two-factors ANOVA test.

FIG. 50 shows erectile responses elicited by cavernosal nerve electrical stimulation in anesthetized diabetic rats after intravenous administration of sildenafil (0.3 mg/kg) and previously treated for 10 days with vehicle (DMSO/PEG 300, 1:1; 12 μL/day s.c.), atenolol or nebivolol (both at 3 and 10 mg/kg/day s.c. for 10 days in DMSO/PEG 300, 1:1 at 12 μL/day rate) after 8 weeks of untreated diabetes. Data are expressed as the mean±SEM of the area under the curve (AUC) of intracavernosal pressure (ICP) increase to cavernosal nerve stimulation (in mm Hg×s) normalized by mean arterial pressure (MAP) values. n indicates the number of animals used. * p<0.05, ** p<0.01 vs No Diabetes, # p<0.05, ‡ p<0.01, § p<0.001 vs No Diabetes+sildenafil, † p<0.05 vs Diabetes and ¥ p<0.01 vs Diabetes+atenolol by a two-factors ANOVA test.

FIG. 51 shows the effects of sustained subcutaneous administration of nebivolol and atenolol (3 and 10 mg/kg/day for 10 days) on serum nitrite plus nitrate (NOx) concentration in diabetic rats after intravenous administration of vehicle (25% glycofurol). Data are expressed as mean±SEM of the NOx concentration in μM. numbers of animals used for determinations are in parenthesis. ** p<0.01, *** p<0.001 vs No diabetes, † p<0.05 vs Diabetes by t-test

FIG. 52 shows the effects of sustained subcutaneous administration of nebivolol and atenolol (3 and 10 mg/kg/day for 10 days) on serum nitrite plus nitrate (NOx) levels in diabetic rats after intravenous administration of sildenafil (0.3 mg/kg). Data are expressed as mean±SEM of the NOx concentration in μM. Numbers of animals used for determinations are in parenthesis. ** p<0.01, *** p<0.001 vs No diabetes, † p<0.05 vs Diabetes by t-test.

FIG. 53 shows the influence of sildenafil (0.3 mg/kg i.v.) on the effects of sustained subcutaneous administration of nebivolol and atenolol (3 and 10 mg/kg/day for 10 days) on serum nitrite plus nitrate (NOx) levels in diabetic rats. Data are expressed as mean±SEM of the NOx concentration in μM. Numbers of animals used for determinations are in parenthesis. ** p<0.01, *** p<0.001 vs No diabetes, † p<0.05 vs Diabetes by t-test.

FIG. 54 shows the effects of sustained subcutaneous administration of nebivolol and atenolol (3 and 10 mg/kg/day for 10 days) on plasma cGMP levels in diabetic rats after intravenous administration of vehicle (25% glycofurol). Data are expressed as mean±SEM of the cGMP concentration in pmol/mL. Numbers of animals used for determinations are in parenthesis.

FIG. 55 shows the effects of sustained subcutaneous administration of nebivolol and atenolol (3 and 10 mg/kg/day for 10 days) on plasma cGMP levels in diabetic rats after intravenous administration of sildenafil (0.3 mg/kg). Data are expressed as mean±SEM of the cGMP concentration in pmol/mL. Numbers of animals used for determinations are in parenthesis.

FIG. 56 shows the influence of sildenafil (0.3 mg/kg i.v.) on the effects of sustained subcutaneous administration of nebivolol and atenolol (3 and 10 mg/kg/day for 10 days) on plasma cGMP levels in diabetic rats. Data are expressed as mean±SEM of the cGMP concentration in pmol/mL. Numbers of animals used for determinations are in parenthesis.

FIG. 57 shows the effects of sustained subcutaneous administration of nebivolol and atenolol (3 and 10 mg/kg/day for 10 days) on cGMP levels in penile tissue from diabetic rats after intravenous administration of vehicle (25% glycofurol). Data are expressed as mean±SEM of the cGMP content normalized by tissue protein content in pmol/g protein. Numbers of animals used for determinations are in parenthesis. † p<0.05 vs Diabetes by t-test.

FIG. 58 shows the effects of sustained subcutaneous administration of nebivolol and atenolol (3 and 10 mg/kg/day for 10 days) on cGMP levels in penile tissue from diabetic rats after intravenous administration of sildenafil (0.3 mg/kg). Data are expressed as mean±SEM of the cGMP content normalized by tissue protein content in pmol/g protein. numbers of animals used for determinations are in parenthesis. ** p<0.01 vs No diabetes, † p<0.05, †† p<0.01 vs Diabetes by t-test.

FIG. 59 shows the influence of sildenafil (0.3 mg/kg i.v.) on the effects of sustained subcutaneous administration of nebivolol and atenolol (3 and 10 mg/kg/day for 10 days) on penile cGMP levels in diabetic rats. Data are expressed as mean±SEM of the cGMP content normalized by tissue protein content in pmol/g protein. Numbers of animals used for determinations are in parenthesis. ** p<0.01 vs No diabetes, † p<0.05, †† p<0.01 vs Diabetes by t-test.

FIG. 60 shows the influence of sildenafil (0.3 mg/kg i.v.) on the effects of sustained subcutaneous administration of nebivolol and atenolol (3 and 10 mg/kg/day for 10 days) on thiobarbituric acid reactive substances (TBARS) levels in sera from diabetic rats. Data are expressed as mean±SEM of malondiahdehyde (MDA) equivalents in ng/mL. Numbers of animals used for determinations are in parenthesis, † vs Diabetes with sildenafil by t-test.

FIG. 61 shows the effects of sustained subcutaneous administration of nebivolol and atenolol (3 and 10 mg/kg/day for 10 days) on thiobarbituric acid reactive substances (TBARS) levels in sera from diabetic rats. Data are expressed as mean±SEM of malondiahdehyde (MDA) equivalents in ng/mL. Numbers of animals used for determinations are in parenthesis. * p<0.01 vs No diabetes, † vs Diabetes by t-test.

### DETAILED DESCRIPTION OF THE INVENTION

In one aspect, the present invention relates to methods for the treatment of sexual dysfunction by administering nebivolol, or a pharmaceutically acceptable salt thereof.

There are many types of sexual dysfunction in males and females. These include, but are not limited to, erectile dysfunction (ED), impotence, premature ejaculation, priapism, ejaculatory incompetence and retarded ejaculation in males and sexual arousal disorder, orgasmic disorder, inhibited orgasm, clitoral dysfunction, female hypoactive sexual desire disorder, vaginismus, dysparcunia, and painful or difficult intercourse in females.

It has now been surprisingly found that nebivolol activates the NO/cGMP pathway, and dilates human penile arteries. Furthermore, nebivolol unexpectedly potentiates normal erectile function in rats. Thus, it is an object of the present invention to treat sexual dysfunction using nebivolol, or a pharmaceutically acceptable salt thereof.

In one embodiment, the nebivolol, or a pharmaceutically acceptable salt thereof, may be used to treat erectile dysfunction in male patients. In another embodiment, the nebivolol or a pharmaceutically acceptable salt thereof may be administered to treat sexual arousal disorder in female patients.

In certain embodiments, the nebivolol is in the form of a hydrochloride salt.

According to some embodiments, the nebivolol, or a pharmaceutically acceptable salt thereof (e.g., nebivolol hydrochloride) may be administered in doses ranging from about 0.1 mg to about 20 mg per day in single or multiple administrations. In some embodiments, the nebivolol, or a pharmaceutically acceptable salt thereof, may be administered in doses ranging from about 1 mg to about 15 mg per day. In certain embodiments, the nebivolol, or a pharmaceutically acceptable salt thereof, may be administered in a daily dose of about 0.5 mg, about 1 mg, about 1.5 mg, about 2 mg, about 2.5 mg, about 3 mg, about 3.5 mg, about 4 mg, about 4.5 mg, about 5 mg, about 5.5. mg, about 6 mg, about 6.5 mg, about 7 mg, about 7.5 mg, about 8 mg, about 8.5 mg, about 9 mg, about 9.5 mg, about 10 mg, about 12.5 mg, about 15 mg, about 17.5 mg or about 20 mg. For example, the nebivolol, or a pharmaceutically acceptable salt thereof, may be administered in a daily dose of about 2.5 mg, about 5 mg, about 10 mg or about 20 mg, for example, in a daily dose of about 2.5 mg, about 5 mg or about 10 mg. In some embodiments, the daily dose of nebivolol, or a pharmaceutically acceptable salt thereof, may be given in a single administration. In other embodiments, the daily dose of nebivolol or a pharmaceutically acceptable salt thereof may be given in multiple administrations. For example, the daily dosage may be divided into one, into two, into three, or into four divided daily doses.

In further embodiments, the nebivolol, or a pharmaceutically acceptable salt thereof, may be administered orally, intravenously, sublingually, or buccally. For example, the nebivolol, or a pharmaceutically acceptable salt thereof, may be administered orally.

Furthermore, it has been unexpectedly found that nebivolol acts synergistically in combination with a PDE-5 inhibitor, such as sildenafil. Nebivolol potentiates the sildenafil-induced relaxation of human corpus cavernosum (HCC) tissue. Furthermore, the combination of nebivolol and sildenafil modulates the cGMP pathway in rats by synergistically elevating the plasma cGMP levels. As used herein, the term sildenafil refers to the base free form of sildenafil and to pharmaceutically acceptable salts thereof, e.g. sildenafil citrate.

Therefore, in a further aspect, the present invention relates to a method of treating a sexual dysfunction by administering nebivolol, or a pharmaceutically acceptable salt thereof, in combination with a second active agent (e.g. a PDE-5 inhibitor). In certain embodiments, the combination of nebivolol or a pharmaceutically acceptable salt thereof and a second active agent may be used to treat erectile dysfunction. In some embodiments, the combination of nebivolol and a second active agent may be used to treat female sexual arousal disorder.

In certain embodiments, the sexual dysfunction may be treated using a combination of nebivolol, or a pharmaceutically acceptable salt thereof, and a PDE-5 inhibitor, or a pharmaceutically acceptable salt thereof. Suitable PDE-5 inhibitors include, but are not limited to, sildenafil, tadalafil, vardenafil, zaprinast, and pharmaceutically acceptable salts thereof (e.g., sildenafil citrate).

Sildenafil is disclosed, for example, in U.S. Pat. Nos. 5,250,534 and 6,469,012. Tadalafil is disclosed, for example, in U.S. Pat. Nos. 5,859,006 and 6,821,975. Vardenafil is disclosed, for example, in U.S. Pat. No. 6,362,178.

In certain embodiments, the combination of nebivolol, or a pharmaceutically acceptable salt thereof, and a PDE-5 inhibitor, or a pharmaceutically acceptable salt thereof, may be administered together, for example, combined as part of a composition. In other embodiments, the combination of nebivolol, or a pharmaceutically acceptable salt thereof, and a PDE-5 inhibitor, or a pharmaceutically acceptable salt thereof, may be administered separately, e.g., sequentially.

In certain embodiments, the sexual dysfunction may be treated using a combination of nebivolol, a pharmaceutically acceptable salt thereof (e.g., nebivolol hydrochloride) and sildenafil, or a pharmaceutically acceptable salt thereof (e.g., sildenafil citrate). In some embodiments, the sildenafil, or a pharmaceutically acceptable salt thereof (e.g., sildenafil citrate) may be administered in doses ranging from about 0.1 mg to about 200 mg per day in single or multiple administrations. In certain embodiments, the sildenafil, or a pharmaceutically acceptable salt thereof, may be administered in doses ranging from about 10 mg to about 100 mg per day. For example, the sildenafil, or a pharmaceutically acceptable salt thereof may be administered in a daily dose of about 5 mg, about 10 mg, about 20 mg, about 25 mg, about 50 mg, about 100 mg, about 150 mg or about 200 mg. In further embodiments, the sildenafil or a pharmaceutically acceptable salt thereof may be administered in a daily dose of about 25 mg, about 50 mg or about 100 mg. For example, the sildenafil or a pharmaceutically acceptable salt thereof may be administered in a daily dose of about 50 mg.

In additional embodiments, the sexual dysfunction may be treated using a combination of nebivolol, or pharmaceutically acceptable salt thereof (e.g., nebivolol hydrochloride) and tadalafil, or a pharmaceutically acceptable salt thereof. In some embodiments, the tadalafil, or a pharmaceutically acceptable salt thereof, may be administered in doses ranging from about 0.1 mg to about 200 mg per day in single or multiple administrations. In some embodiments, the tadalafil, or a pharmaceutically acceptable salt thereof, may be administered in doses ranging from about 1 mg to about 100 mg per day. In other embodiments, the tadalafil, or a pharmaceutically acceptable salt thereof, may be administered in a daily dose of about 2.5 mg, about 5 mg, about 10 mg, 20 mg, about 25 mg, about 50 mg or about 100 mg. In certain embodiments, the tadalafil, or a pharmaceutically acceptable salt thereof, may be administered in daily doses of about 5 mg, about 10 mg or about 20 mg. For example, the tadalafil, or a pharmaceutically acceptable salt thereof, may be administered in daily doses of about 10 mg.

In further embodiments, the sexual dysfunction may be treated using a combination of nebivolol, or a pharmaceutically acceptable salt thereof (e.g., nebivolol hydrochloride) and vardenafil, or a pharmaceutically acceptable salt thereof. In some embodiments, the vardenafil, or a pharmaceutically acceptable salt thereof, may be administered in doses ranging from about 0.1 mg to about 200 mg per day in single or multiple administrations. In some embodiments, the vardenafil, or a pharmaceutically acceptable salt thereof, may be administered in doses ranging from about 1 mg to about 100 mg per day. In other embodiments, the vardenafil, or a pharmaceutically acceptable salt thereof, may be administered in a daily dose of about 2.5 mg, about 5 mg, about 10 mg, about 20 mg, about 25 mg, about 50 mg or about 100 mg. In certain embodiments, the vardenafil, or a pharmaceutically acceptable salt thereof, may be administered in a daily dose of about 2.5 mg, about 5 mg, about 10 mg or about 20 mg, such as in a daily dose of about 10 mg.

In certain embodiments, the sexual dysfunction may be treated using a combination of nebivolol, or a pharmaceutically acceptable salt thereof (e.g., nebivolol hydrochloride) and zaprinast, or a pharmaceutically acceptable salt thereof. In some embodiments, the zaprinast, or a pharmaceutically acceptable salt thereof, may be administered in doses ranging from about 0.1 mg to about 200 mg per day in single or multiple administrations. In some embodiments, the zaprinast, or a pharmaceutically acceptable salt thereof, may be administered in doses ranging from about 10 mg to about 100 mg per day. In other embodiments, the zaprinast, or a pharmaceutically acceptable salt thereof, may be administered in a daily dose of about 2.5 mg, about 5 mg, about 10 mg, about 20 mg, about 25 mg, about 50 mg, about 100 mg, about 150 mg or about 200 mg. In certain embodiments, the zaprinast, or a pharmaceutically acceptable salt thereof may be administered in a daily dose of about 50 mg.

According to a further aspect, the present invention relates to a method of treating a sexual dysfunction in a patient in need thereof comprising administering to the patient a therapeutically acceptable amount of nebivolol, or a pharmaceutically acceptable salt thereof, and a PDE-5 inhibitor (e.g., sildenafil), wherein the nebivolol, or a pharmaceutically acceptable salt thereof, enhances the PDE-5 inhibitor (e.g., sildenafil) induced-relaxation of human corpus cavernosum tissue by at least about 2.5%, as compared to treatment with a PDE-5 inhibitor (e.g., sildenafil) alone, such as at least about 5%, at least about 7.5%, at least about 10%, at least about 12.5%, at least about 15%, at least about 17.5%, at least about 20%, at least about 22.5%, at least about 25%, at least about 27.5%, at least about 30%, at least about 32.5%, at least about 35%, at least about 37.5%, or even at least about 40% as compared to treatment with a PDE-5 inhibitor (e.g., sildenafil) alone.

According to a further aspect, the present invention relates to a method of enhancing PDE-5 inhibitor (e.g., sildenafil) induced relaxation of human corpus cavernosum tissue in a patient receiving a PDE-5 inhibitor (e.g., sildenafil) comprising administering to the patient a therapeutically acceptable amount of nebivolol, or a pharmaceutically acceptable salt thereof, wherein the PDE-5 inhibitor (e.g., sildenafil) induced-relaxation of human corpus cavemosum tissue is enhanced by at least about 2.5%, as compared to treatment with a PDE-5 inhibitor (e.g., sildenafil) alone, such as at least about 5%, at least about 7.5%, at least about 10%, at least about 12.5%, at least about 15%, at least about 17.5%, at least about 20%, at least about 22.5%, at least about 25%, at least about 27.5%, at least about 30%, at least about 32.5%, at least about 35%, at least about 37.5%, or even at least about 40% as compared to treatment with a PDE-5 inhibitor (e.g., sildenafil) alone.

According to a further aspect, the present invention relates to a method of enhancing PDE-5 inhibitor (e.g., sildenafil) induced-relaxation of human corpus cavernosum tissue, comprising administering to the tissue a therapeutically acceptable amount of nebivolol, or a pharmaceutically acceptable salt thereof, and a PDE-5 inhibitor (e.g., sildenafil), wherein the PDE-5 inhibitor (e.g., sildenafil) induced-relaxation of human corpus cavernosum tissue is enhanced by at least about 2.5%, as compared to the relaxation level that occurs with the PDE-5 inhibitor (e.g., sildenafil) administration alone, such as at least about 5%, at least about 7.5%, at least about 10%, at least about 12.5%, at least about 15%, at least about 17.5%, at least about 20%, at least about 22.5%, at least about 25%, at least about 27.5%, at least about 30%, at least about 32.5%, at least about 35%, at least about 37.5%, or even at least about 40% as compared to the relaxation level that occurs with PDE-5 inhibitor (e.g., sildenafil) administration alone.

According to a further aspect, the present invention relates to a method of treating a sexual dysfunction in a patient in need thereof comprising administering to the patient a therapeutically acceptable amount of nebivolol, or a pharmaceutically acceptable salt thereof, wherein the nebivolol, or a pharmaceutically acceptable salt thereof, dilates human penile resistance arteries by at least about 2.5%, such as at least about 5%, at least about 7.5%, at least about 10%, at least about 12.5%, at least about 15%, at least about 17.5%, at least about 20%, at least about 22.5%, at least about 25%, at least about 27.5%, at least about 30%, at least about 32.5%, at least about 35%, at least about 37.5%, at least about 40%, at least about 45%, at least about 50%, at least about 55% or even at least about 60%.

According to a further aspect, the present invention relates to a method of dilating human penile resistance arteries in a patient in need of treatment for a sexual dysfunction, comprising administering to the patient a therapeutically acceptable amount of nebivolol, or a pharmaceutically acceptable salt thereof, wherein the human penile resistance arteries are dilated by at least about 2.5%, such as at least about 5%, at least about 7.5%, at least about 10%, at least about 12.5%, at least about 15%, at least about 17.5%, at least about 20%, at least about 22.5%, at least about 25%, at least about 27.5%, at least about 30%, at least about 32.5%, at least about 35%, at least about 37.5%, at least about 40%, at least about 45%, at least about 50%, at least about 55% or even at least about 60%.

According to a further aspect, the present invention relates to a method of enhancing PDE-5 inhibitor (e.g., sildenafil) mediated-dilation of human penile resistance arteries in a patient comprising administering to the patient a therapeutically acceptable amount of nebivolol, or a pharmaceutically acceptable salt thereof, wherein dilation of the human penile resistance arteries is enhanced by at least about 2.5%, such as at least about 5%, at least about 7.5%, at least about 10%, at least about 12.5%, at least about 15%, at least about 17.5%, at least about 20%, at least about 22.5%, at least about 25%, at least about 27.5%, at least about 30%, at least about 32.5%, at least about 35%, at least about 37.5%, at least about 40%, at least about 45%, at least about 50%, at least about 55%, at least about 60%, at least about 65%, at least about 70%, at least about 75% at least about 80%, at least about 85% or even at least about 90% as compared to the dilation level that occurs with PDE-5 inhibitor (e.g., sildenafil) administration alone.

According to a further aspect, the present invention relates to a method of treating a sexual dysfunction in a patient in need thereof comprising administering to the patient a therapeutically acceptable amount of nebivolol, or a pharmaceutically acceptable salt thereof, and a PDE-5 inhibitor (e.g., sildenafil), wherein the nebivolol, or a pharmaceutically acceptable salt thereof, enhances PDE-5 inhibitor (e.g., sildenafil) dilation of human penile resistance arteries by at least about 2.5%, such as at least about 5%, at least about 7.5%, at least about 10%, at least about 12.5%, at least about 15%, at least about 17.5%, at least about 20%, at least about 22.5%, at least about 25%, at least about 27.5%, at least about 30%, at least about 32.5%, at least about 35%, at least about 37.5%, at least about 40%, at least about 45%, at least about 50%, at least about 55%, at least about 60%, at least about 65%, at least about 70%, at least about 75% at least about 80%, at least about 85% or even at least about 90% as compared to the dilation level that occurs with PDE-5 inhibitor (e.g., sildenafil) administration alone.

According to a further aspect, the present invention relates to a method of treating a sexual dysfunction in a patient in need thereof comprising administering to the patient a therapeutically acceptable amount of nebivolol, or a pharmaceutically acceptable salt thereof, wherein the plasma and/or tissue cGMP levels in the patient are increased by at least about 105%, such as by at least about 110%, by at least about 115%, by at least about 120%, by at least about 125%, by at least about 130%, by at least about 135%, by at least about 140% or by at least about 150%.

According to a further aspect, the present invention relates to a method of treating a sexual dsyfunction in a patient in need thereof comprising administering to the patient a therapeutically acceptable amount of nebivolol, or a pharmaceutically acceptable salt thereof, wherein the plasma and/or tissue cGMPlevels in the patient are increased by at least about 150%, such as at least about 175%, at least about 180%, at least about 190%, at least about 200%, at least about 210%, at least about 220%, at least about 225%, at least about 230%, at least about 240% or even at least about 250%.

According to a further aspect, the present invention relates to a method of treating a sexual dsyfunction in a patient in need thereof comprising administering to the patient a therapeutically acceptable amount of nebivolol, or a pharmaceutically acceptable salt thereof, and sildenafil, wherein the plasma and/or tissue cGMP levels in the patient are increased by at least about 300%, such as at least about 310%, at least about 320%, 325%, at least about 330%, at least about 340%, at least about 350%, at least about 360%, at least about 370%, at least about 375%, at least about 380%, at least about 390% or even at least about 400%.

In some embodiments, the nebivolol, or a pharmaceutically acceptable salt thereof, and the type 5 phosphodiesterase (PDE-5) inhibitor, or a pharmaceutically acceptable salt thereof, may be administered orally, intravenously, sublingually, or buccally. For example, the nebivolol, or a pharmaceutically acceptable salt thereof, and the type 5 phosphodiesterase (PDE-5) inhibitor, or a pharmaceutically acceptable salt thereof may be administered orally.

The active ingredient(s) can be administered alone or as an active ingredient of a formulation. Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), (current edition).

The mode of administration and dosage forms is closely related to the therapeutic amounts of the compounds or compositions which are desirable and efficacious for the given treatment application.

Suitable dosage forms include but are not limited to oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterille administration, and other dosage forms for systemic delivery of active ingredients. For example, the active ingredient(s) may be administered orally.

To prepare such pharmaceutical dosage forms, the active ingredient is typically admixed with a pharmaceutical carrier according to conventional pharmaceutical compounding techniques. The carrier may take a wide variety of forms depending on the form of preparation desired for administration.

In preparing the compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. Due to their ease in administration, tablets and capsules represent the most advantageous oral dosage unit form. If desired, tablets may be sugar coated or enteric coated by standard technique.

For parenteral formulations, the carrier will usually comprise sterile water, though other ingredients, for example, ingredients that aid solubility or for preservation, may be included. Injectable solutions may also be prepared in which case appropriate stabilizing agents may be employed.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Treatment methods of the present invention using formulations suitable for oral administration may be presented as discrete units such as capsules, cachets, tablets, or lozenges, each containing a predetermined amount of the active ingredient as a powder or granules. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration usually comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations may further include one or more accessory ingredient(s) selected from diluents, buffers, flavoring agents, binders, disintegrants, surface active agents, thickeners, lubricants, preservatives (including antioxidants), and the like.

The formulations can have immediate release, sustained release, delayed-onset release or any other release profile known to one skilled in the art.

The desired dose may be administered as one or more daily sub dose(s) administered at appropriate time intervals throughout the day, or alternatively, in a single dose, for example, for morning or evening administration. For example, the daily dosage may be divided into one, into two, into three, or into four divided daily doses. The duration of the treatment may be decades, years, months, weeks, or days, as long as the benefits persist.

### Definitions

The term “pharmaceutically acceptable” means biologically or pharmacologically compatible for in vivo use in animals or humans, and preferably means approved by a regulatory agency of the Federal or a State government or listed in the U.S. Pharmacopeia or other generally recognized pharmacopeia for use in animals, and more particularly in humans.

Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride salt, a hydrobromide salt or a citrate salt.

The term “treating” means to relieve, alleviate, delay, reduce, reverse, improve, manage or prevent at least one symptom of a condition in a subject. The term “treating” may also mean to arrest, delay the onset (i.e., the period prior to clinical manifestation of a disease) and/or reduce the risk of developing or worsening a condition.

An “effective amount” means the amount of a compound that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

A subject or patient in whom administration of the therapeutic compound is an effective therapeutic regimen for a disease or disorder is preferably a human, but can be any animal, including a laboratory animal in the context of a trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%.

### EXAMPLES

The following examples are given as particular embodiments of the invention and to demonstrate the advantages thereof. It is understood that the examples are given by way of illustration and are not intended to limit the specification or the claims that follow in any manner.

### Example 1

The effect of nebivolol on mean arterial pressure (MAP) and heart rate (HR) in rats was determined.

To determine blood pressure and heart rate, Sprague-Dawley rats (250-400 g) were anesthetized with ketolar and diazepam. The right carotid artery was catheterized for constant blood pressure and heart rate measurement by means of a pressure transducer connected to a PowerLab data acquisition system (ADInstruments). The left external jugular vein was catheterized for saline or drug infusion.

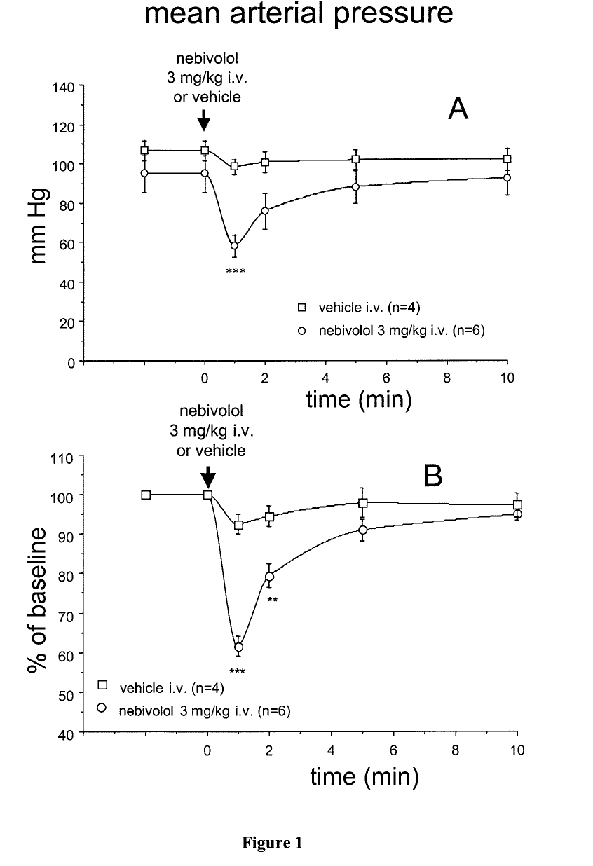

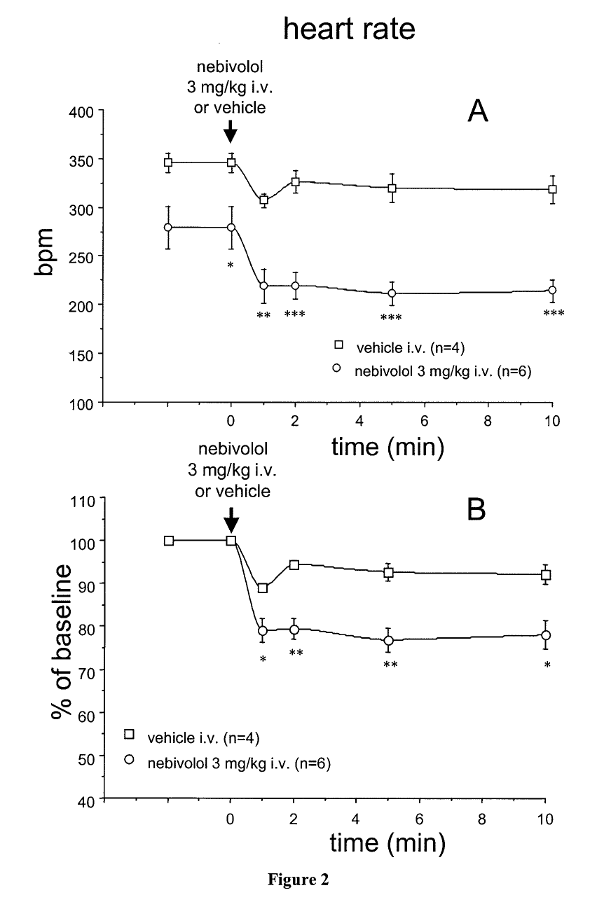

3 mg/kg nebivolol (n=6) or vehicle (50% glycofurol; n=4) was administered intravenously in anesthetized rats and MAP and HR were measured. Nebivolol caused a significant hypotensive effect and a significant reduction of heart rate (See FIGS. 1 and 2).

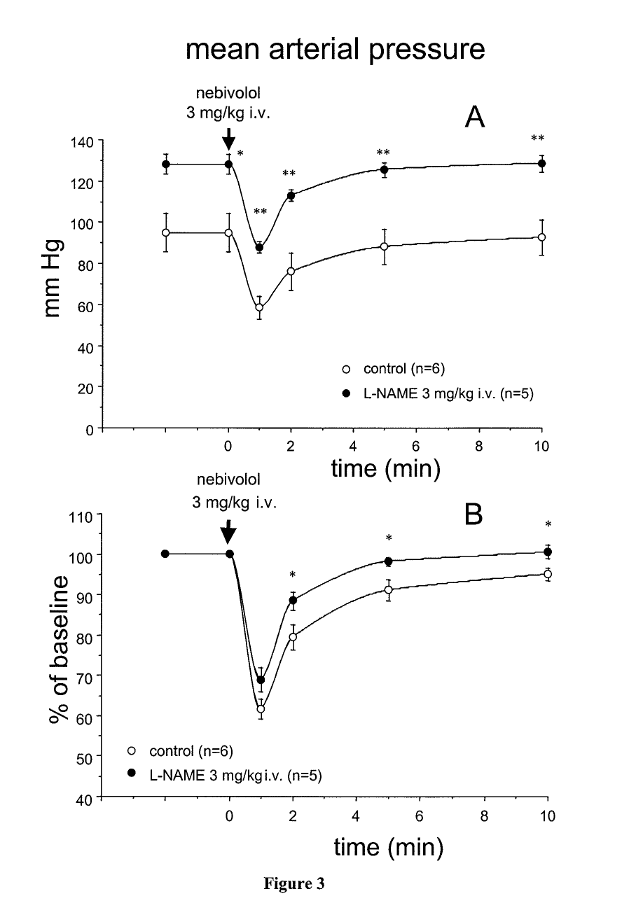

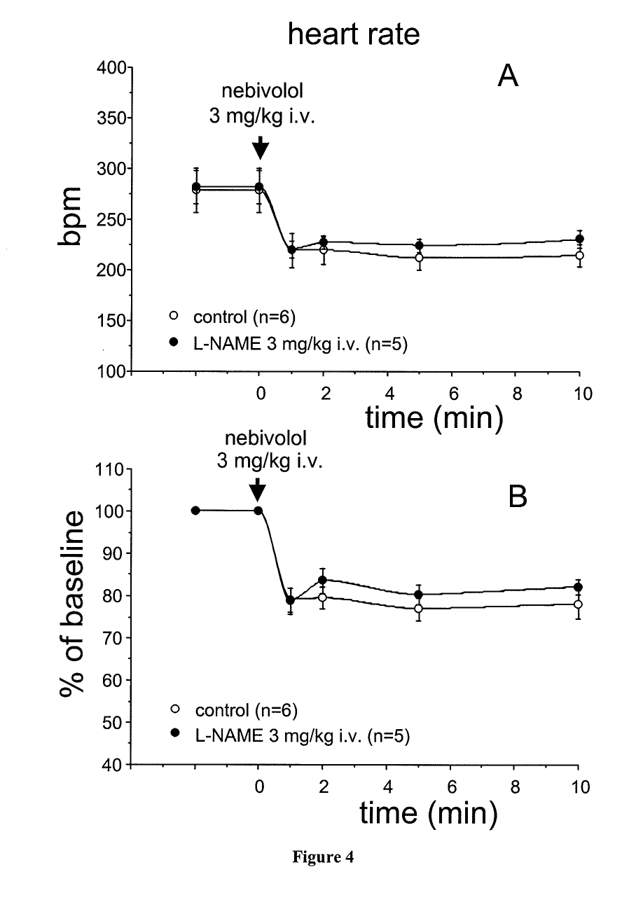

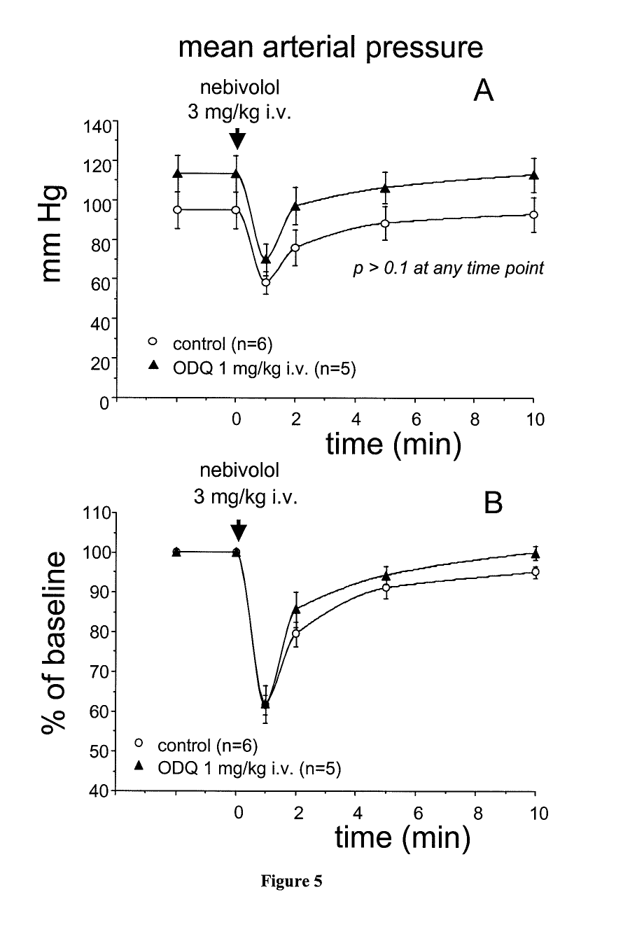

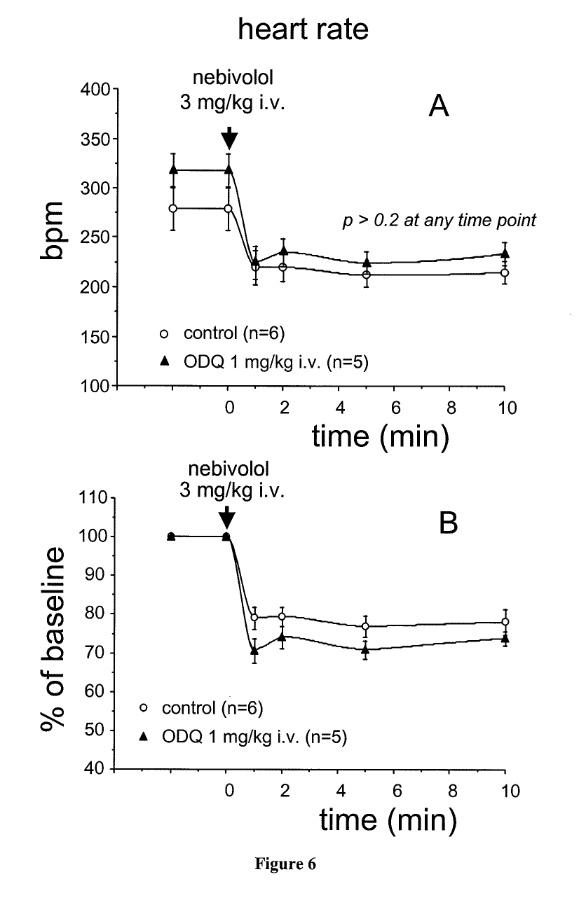

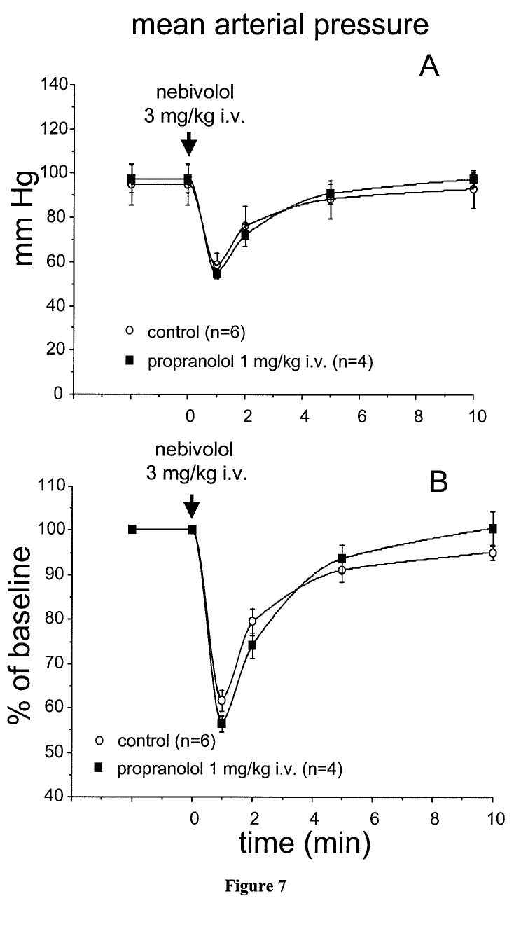

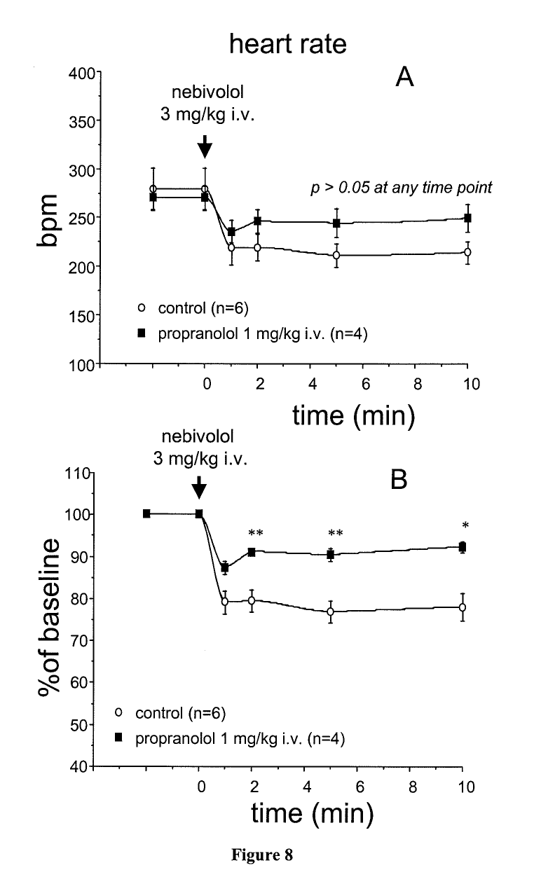

Nebivolol induced hypotension was further investigated by inhibiting the modulators of NO/cGMP pathway, in particular, nitric oxide synthase (NOS) and guanylyl cyclase (GC). The ability of nebivolol to reduce MAP was significantly reduced after treatment with the NOS inhibitor, N_{ω}-nitro-L-arginine methyl ester (L-NAME; 3 mg/kg; i.v.; n=5) (See FIG. 3). L-NAME did not affect nebivolol induced reduction of heart rate (See FIG. 4). Inhibition of guanylyl cyclase with 1-H-(1,2,4)oxadiazolo(4,3-alpha)quinoxaline-1-one (ODQ; 1 mg/kg, i.v.; n=5) did not significantly modify nebivolol-induced effects (See FIGS. 5 and 6). Blockade of β-adrenoceptors with propranolol (1 mg/kg, i.v.; n=4) significantly reduced the impact of nebivolol on heart rate without affecting the nebivolol-induced hypotension (See FIGS. 7 and 8).

### Example 2

The effect of nebivolol in combination with the type 5 phosphodiesterase (PDE-5) inhibitor sildenafil on hypotension and heart rate was studied in rats using the methods described above.

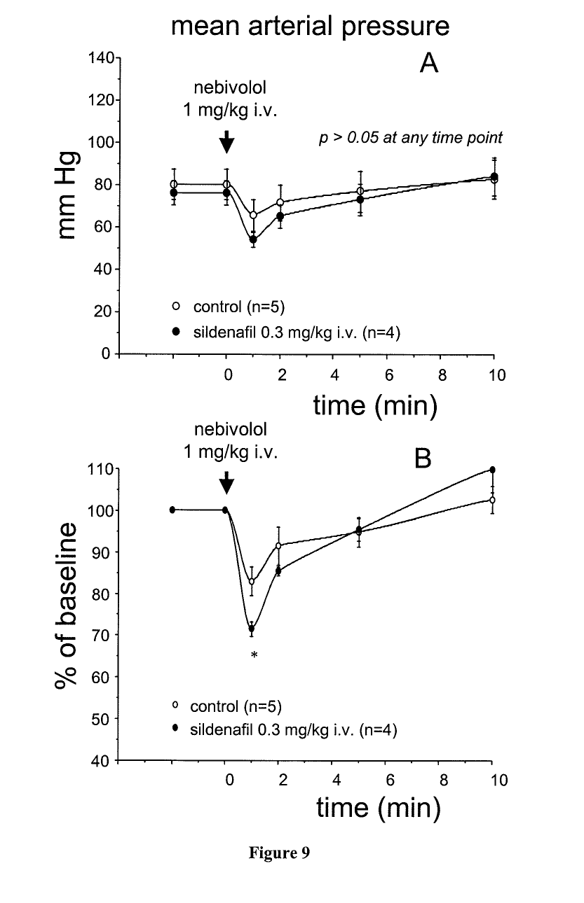

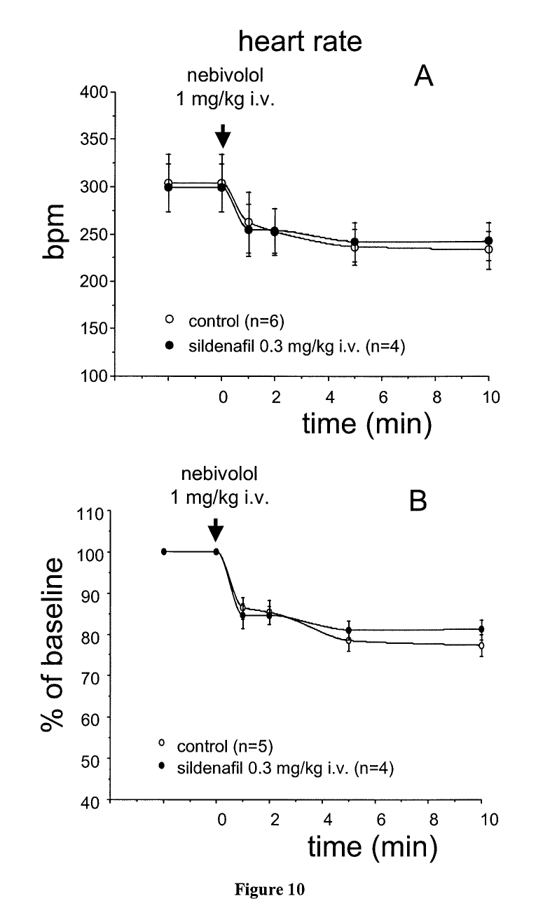

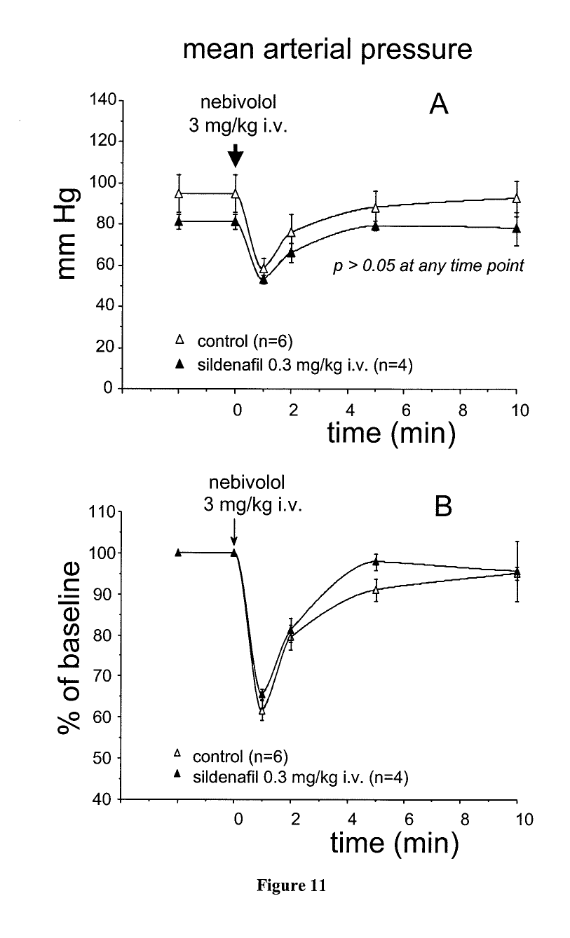

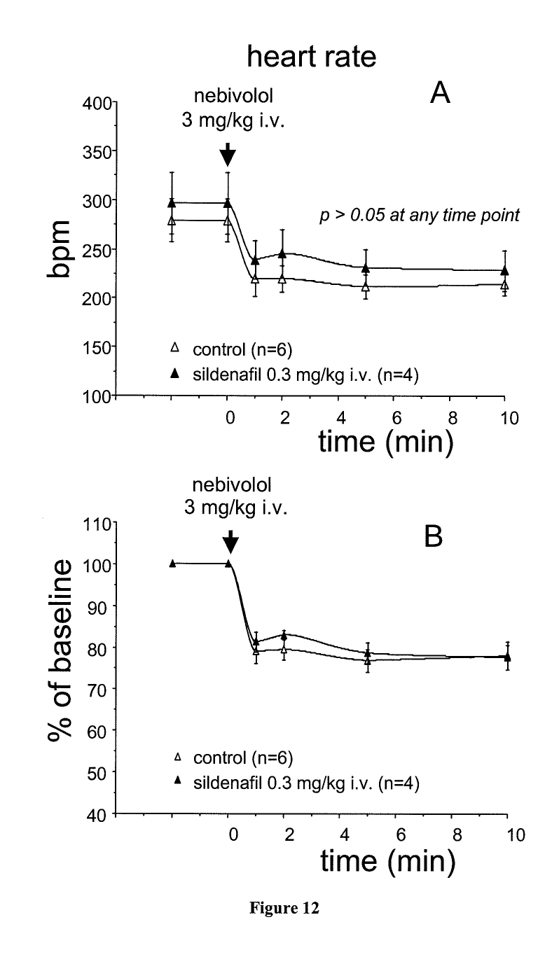

A lower dose of nebivolol (1 mg/kg, i.v.) produced hypotensive and bradycardic effects of moderate intensity. The blood pressure drop caused by 1 mg/kg nebivolol was significantly enhanced when the animals were pre-treated with the PDE-5 inhibitor sildenafil (0.3 mg/kg, i.v.; n=4). The reduction of heart rate caused by 1 mg/kg nebivolol was not modified by PDE-5 inhibition (See FIGS. 9 and 10). Sildenafil did not significantly alter the effects caused by the higher dose of nebivolol (3 mg/kg, i.v.) on blood pressure and heart rate (See FIGS. 11 and 12).

### Example 3

The effects of nebivolol and sildenafil on serum nitrite and nitrate concentration (NOx) and plasma cGMP in rats were studied.

Total NO derivatives (nitrites plus nitrates) were measured in pre-filtered (10,000 MW pore size) serum samples from the studied rats by the Griess colorimetric method, using a commercial kit for nitrite and nitrate determination from Cayman Chemical Co. (Ann Arbor, Mich.). Serum concentrations of NOx were determined 10 minutes after nebivolol administration.

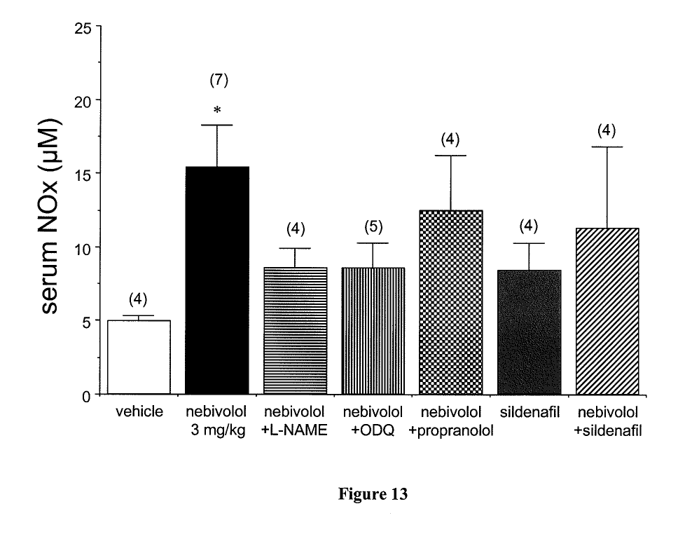

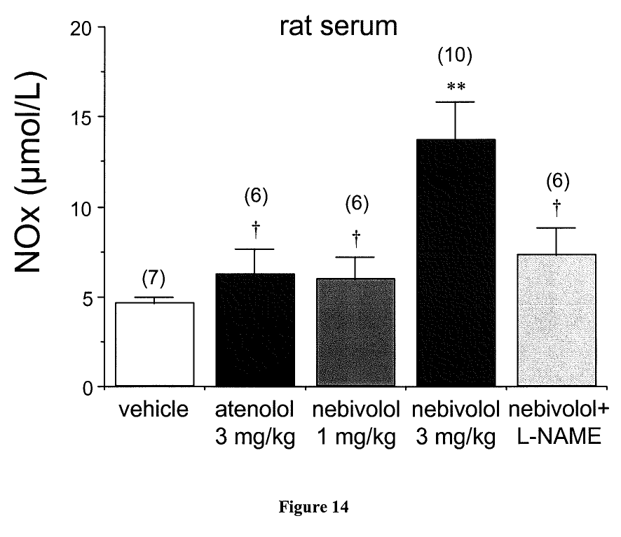

Rat serum concentration of NO derivatives (NOx) after intravenous administration of nebivolol (3 mg/kg) was significantly higher than after vehicle (50% glycofurol) administration (See FIG. 13). Nebivolol caused a three-fold increase of the concentration of NOx in serum compared to the vehicle (15.4±2.8 vs 4.9±0.4 μM, p<0.05). Treatment with the NOS inhibitor L-NAME (3 mg/kg) or the guanylyl cyclase inhibitor ODQ (1 mg/kg), but not propranolol (1 mg/kg), prevents this effect. Sildenafil did not alter NOx levels or affect nebivolol-induced effects on NOx serum levels (See FIG. 13). Intravenous administration of the β-blocker atenolol (3 mg/kg, i.v.; n=6) did not significantly alter the NOx content in rat serum. (See FIG. 14).

To determine the plasma cGMP content, Sprague-Dawley rats were anesthetized with urethane (1.25 g/kg). The right carotid artery was catheterized for constant blood pressure and heart rate measurement by means of a pressure transducer connected to a PowerLab data acquisition system (ADInstruments). The left external jugular vein was catheterized for saline or drug infusion. After administering the respective treatments, rat plasma samples were collected, immediately frozen and stored at −80° C. until extraction for cyclic nucleotide assay. cGMP was determined by ELISA using a kit from Cayman Chemical Co. Plasmatic cGMP levels were determined 10 minutes after nebivolol administration.

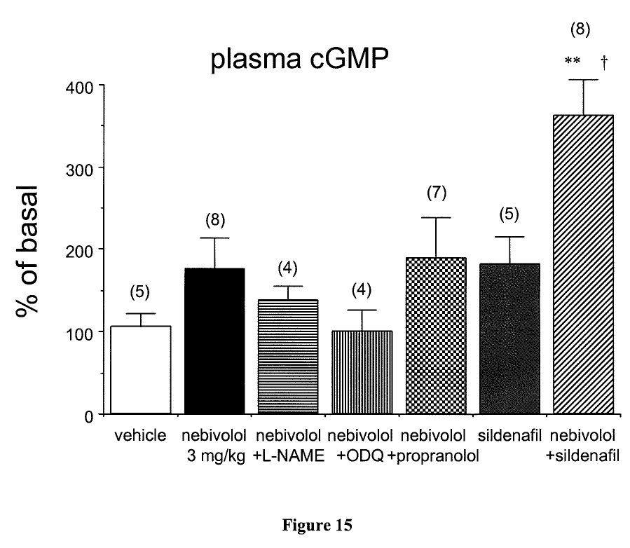

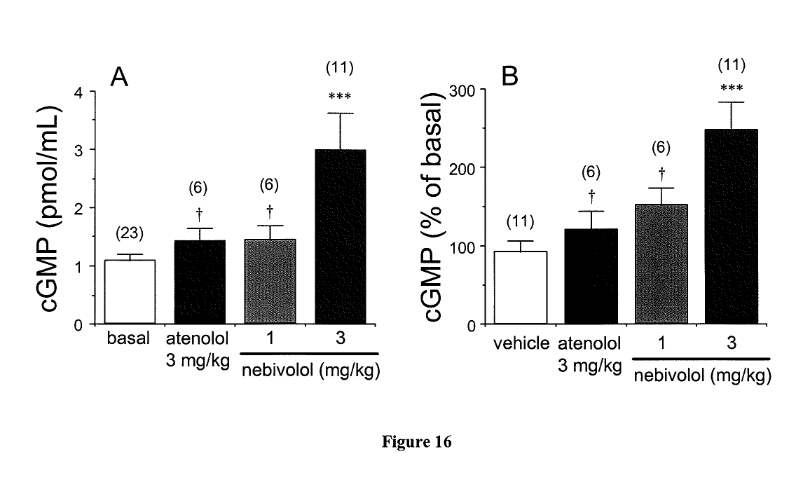

Nebivolol (3 mg/kg, i.v.; n=8) produced an increase in plasma cGMP levels (177±38%) (See FIG. 15). This increase was slightly reduced after administration of L-NAME (3 mg/kg) or ODQ (1 mg/kg), but was not influenced by propranolol (1 mg/kg). The β-blocker atenolol (3 mg/kg; n=6) did not significantly modify plasma cGMP levels in rats. (See FIG. 16).

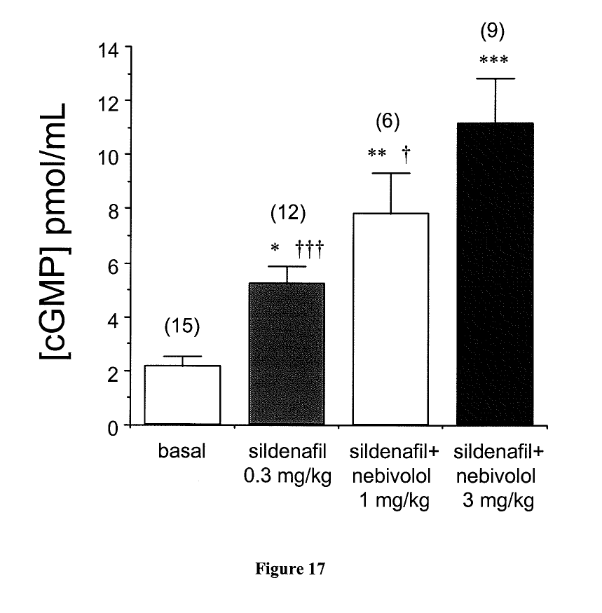

Intravenous administration of sildenafil (0.3 mg/kg) augmented cGMP up to similar levels than nebivolol (3 mg/kg). (See FIG. 14). Combined administration of sildenafil (0.3 mg/kg) and nebivolol (3 mg/kg) resulted in a marked increase of cGMP levels in plasma. The concentration obtained after the treatment of sildenafil in combination with nebivolol was significantly higher than that observed in vehicle treated rats or in rats individually treated with sildenafil or nebivolol (See FIG. 15). A significant further increase of cGMP levels was observed after combining sildenafil with nebivolol at 1 and 3 mg/kg, an effect which was dose-dependent (FIG. 17). The increase in cGMP levels obtained upon using a combination of nebivolol plus sildenafil is much larger than expected for administration of the individual compounds; therefore, these compounds interact synergistically. Thus, it has been unexpectedly found that a combination of sildenafil and nebivolol synergistically increases plasma cGMP levels.

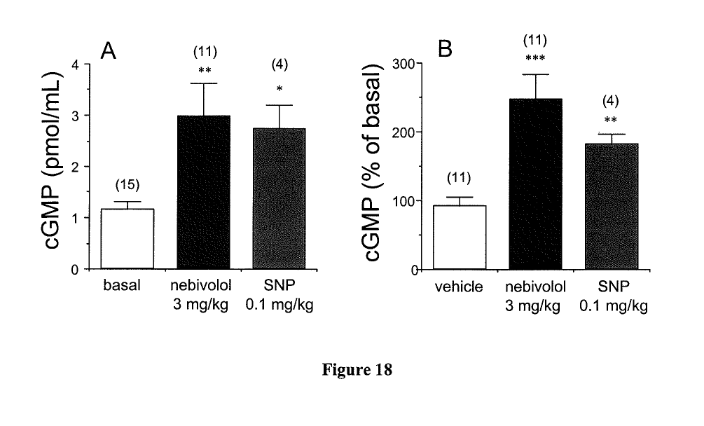

Administration of the NO donor sodium nitroprusside (SNP, 0.1 mg/kg; i.v.; n=4) resulted in a cGMP rise in rat plasma. This increase was comparable to that obtained after administering nebivolol (3 mg/kg; i.v.) (See FIG. 18).

### Example 4

The effects of nebivolol administration on rat erectile function were studied. Erectile responses in rats were evaluated by measuring intracavernosal pressure (ICP) increases to cavernosal nerve electrical stimulation.

To measure ICP, rats (Sprague-Dawley) were anesthetized with ketolar and diazepam. The surgical procedure included dissection and isolation of the right cavernous nerve through an abdominal midline incision and exposure of penile crura through a transverse perineal incision. ICP measurements were permitted by insertion of a 23-gauge needle into the right crus. The right carotid artery and left external jugular vein were catheterized for constant blood pressure measurement and saline infusion, respectively. Electrostimulation was performed with a delicate platinum bipolar hook electrode connected to a stimulator and current amplifier. Parameters of electrical stimulation included pulses with a duration of 0.3 ms and 1.5 mA of current intensity for 1 min. Frequency-response curves were performed by applying stimulation at 1, 3 and 10 Hz with a lapse of 3 min between each increase of frequency.

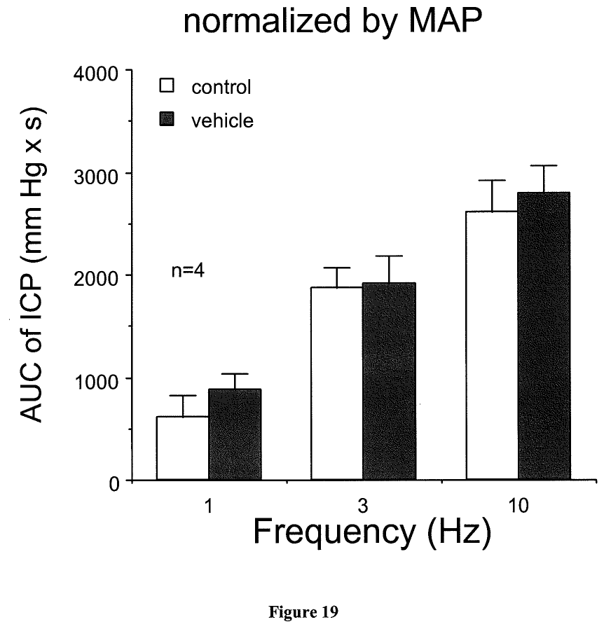

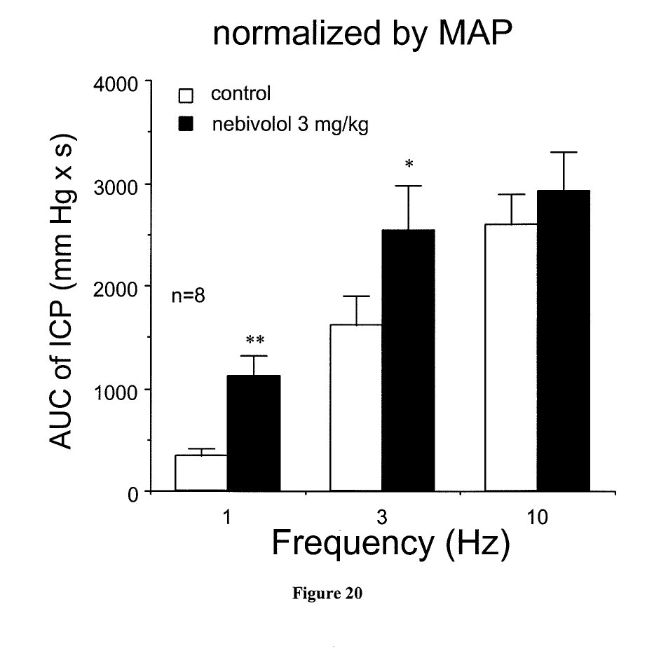

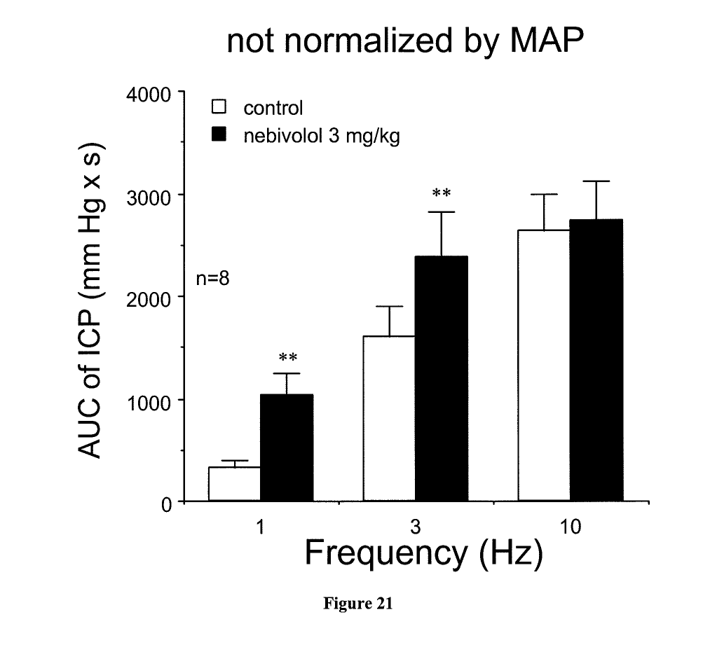

Treatment with nebivolol (3 mg/kg; i.v.) significantly potentiated erectile responses in rats (AUC of ICP at 3 Hz 2544±440 vs. 1612±294 mmHg×s, p<0.05). Nebivolol induced potentiation was seen irrespective of the effects of nebivolol on blood pressure (See FIGS. 19-21).

### Example 5

The effects of nebivolol on the relaxation of isolated human corpus cavernosal tissue (HCC) and penile resistance arteries (HPRA) were determined.

Specimens of human corpus cavernosum were obtained from organ donors and from impotent men at the time of penile prosthesis implantation. Tissues were placed in ice-cold M-400 solution (pH 7.4; 400 mOsm/kg, composition in w/v: 4.19% manitol, 0.2% KH2PO4, 0.97% K₂HPO₄.3H₂O, 0.11% KCl and 0.08% NaHCO₃) at the time of removal and transported to the laboratory for utilization within 16 h.

Strips of human corpus cavemosum were mounted on force transducers in 8 ml organ baths (37° C.) containing physiological salt solution (PSS) continuously bubbled with 95% O₂/5% CO₂ mixture to maintain a pH of 7.4. Strips were contracted with the thromboxane receptor agonist U46619 (1-3 nM) and relaxation responses were evaluated by cumulative additions of compounds to the chambers.

Nebivolol (10 μM) potentiated endothelium-dependent and sildenafil-induced relaxations of HCC. Nebivolol (1 nM to 10 μM) also produced endothelium-dependent vasodilation of HPRA (Eₘₐₓ 59.1±10.3%, EC₅₀ 24.4 nM). Thus, it has been surprisingly found that nebivolol enhances the relaxation of HCC.

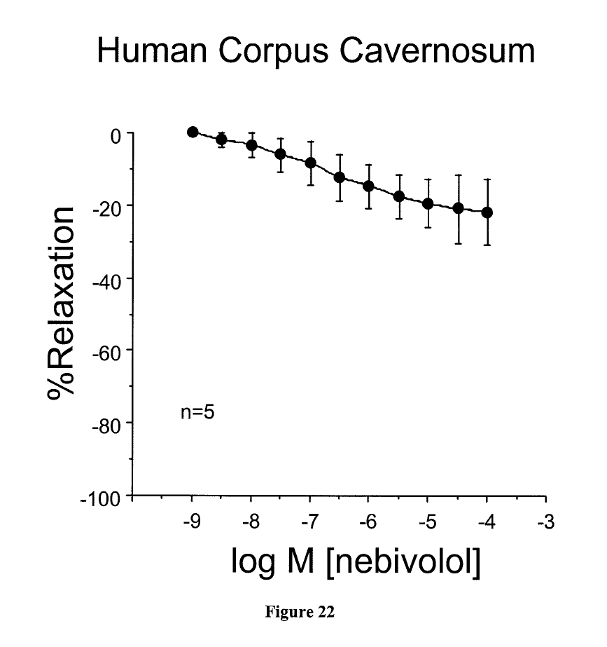

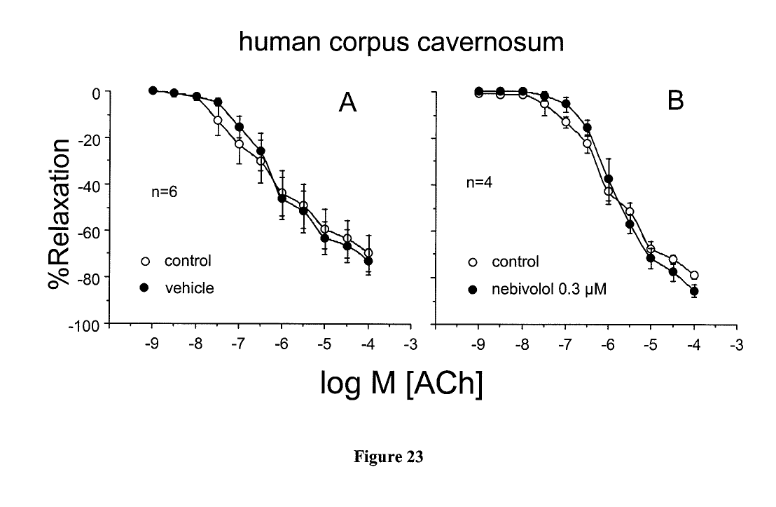

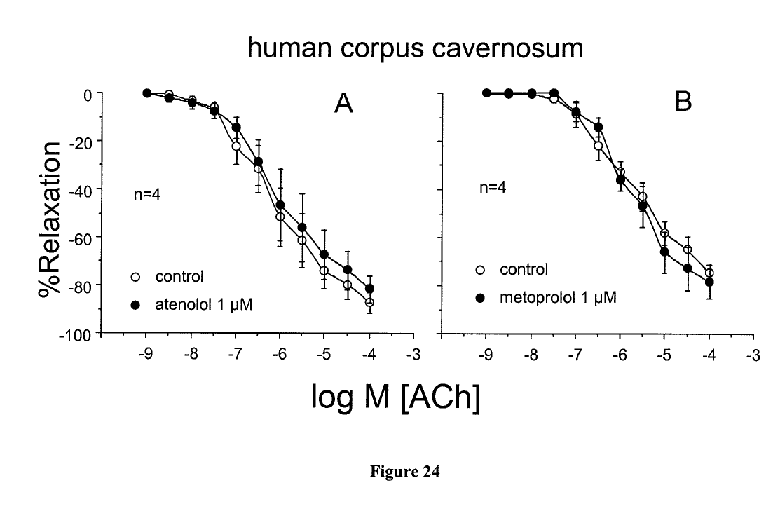

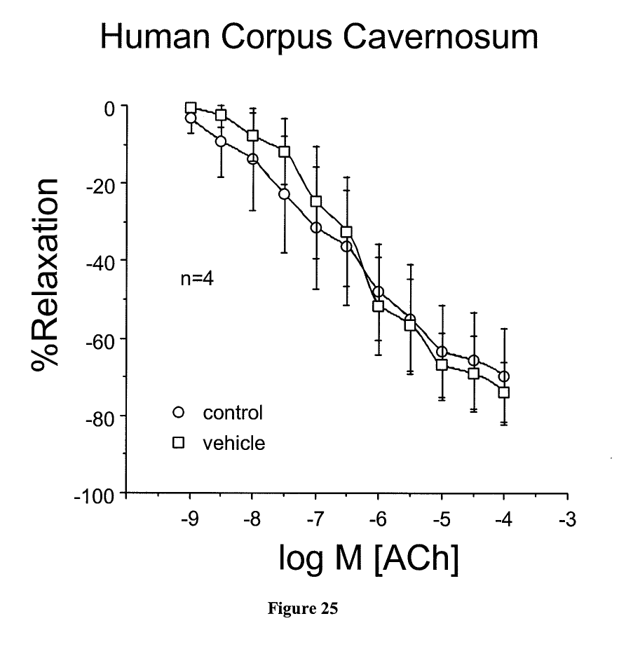

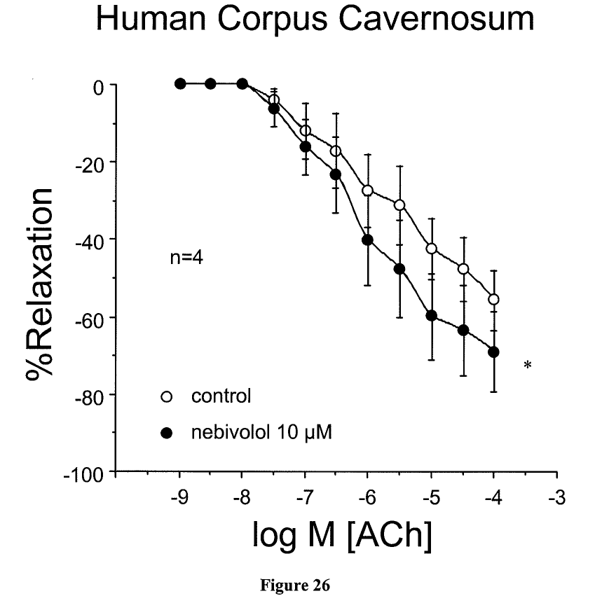

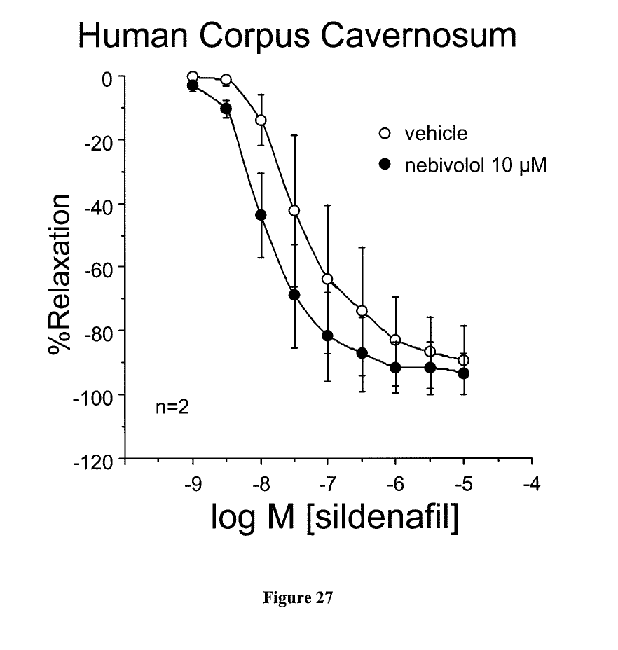

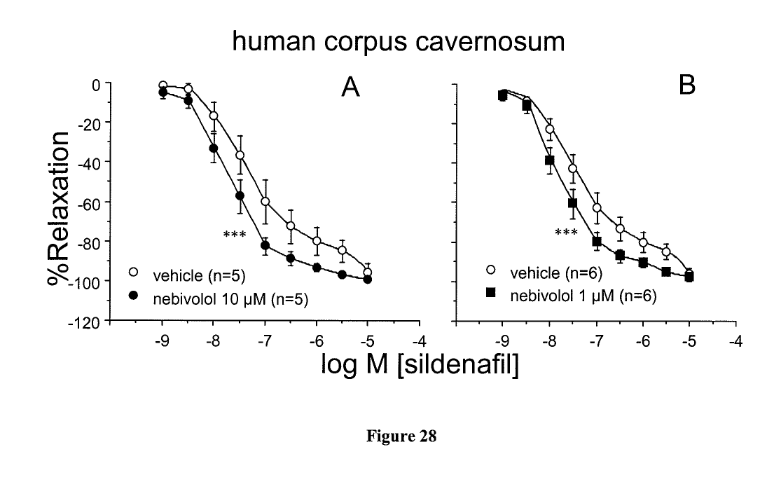

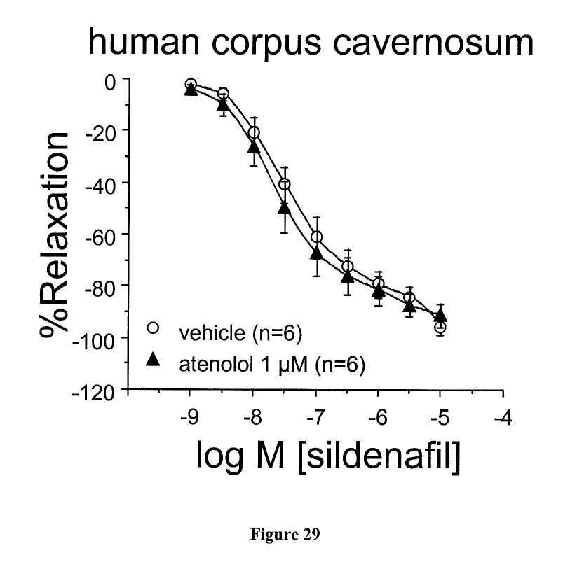

In human corpus cavernosum strips contracted with a thromboxane analog (U46619, 1-3 nM), the cumulative addition of nebivolol (1 nM to 0.1 mM) but not vehicle caused a modest relaxant effect (See FIG. 22). Acetylcholine (ACh)-induced endothelium-dependent relaxation of human corpus cavernosum (HCC) strips was not significantly modified by metoprolol (1 μM), atenolol (1 μM) or nebivolol (0.3 μM) (See FIGS. 23 and 24). Relaxations induced by acetylcholine (ACh) in U46619-contracted HCC were potentiated by pre-treatment with nebivolol (10 μM) but not vehicle (See FIGS. 25 and 26). Nebivolol (10 μM) also showed relaxations induced by sildenafil in HCC contracted with U46619 (See FIG. 27). The PDE5 inhibitor sildenafil (1 nm to 10 μM) caused concentration dependent relaxations of HCC strips contracted with U46619. These relaxations were significantly potentiated by treating the tissues with nebivolol at 1 and 10 μM and were not affected by atenolol (1 μM) (See FIGS. 28 and 29).

To determine the resistance of penile small arteries, helicine arteries (lumen diameter 150-400 μm) which are the terminal branches of deep penile arteries, were dissected by carefully removing the adhering trabecular tissue, and arterial ring segments (2 mm long) were subsequently mounted on two 40 μm wires on microvascular double Halpern-Mulvany myographs (J.P. Trading, Aarhus, Denmark) for isometric tension recordings. The chambers were filled with PSS continuously bubbled with 95% O₂/5% CO₂ mixture to maintain a pH of 7.4. The arteries were contracted with 1 μM U46619 (1-3 nM) and relaxation responses were evaluated by cumulative additions of compounds to the chambers.

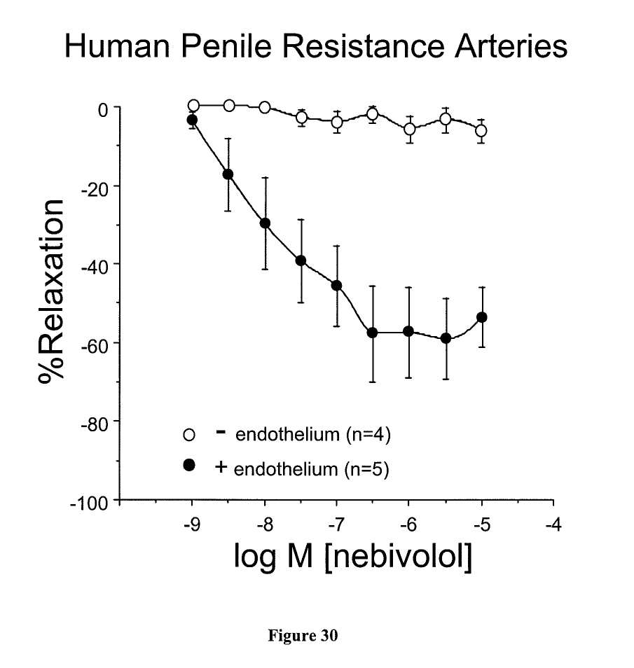

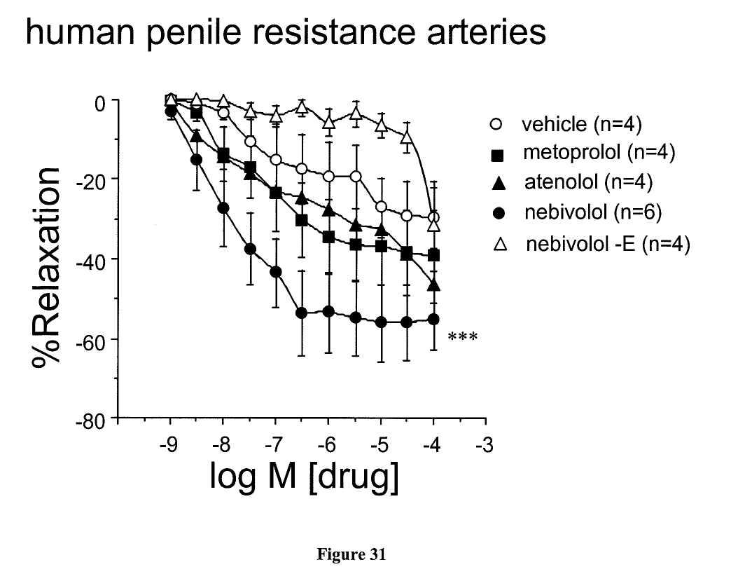

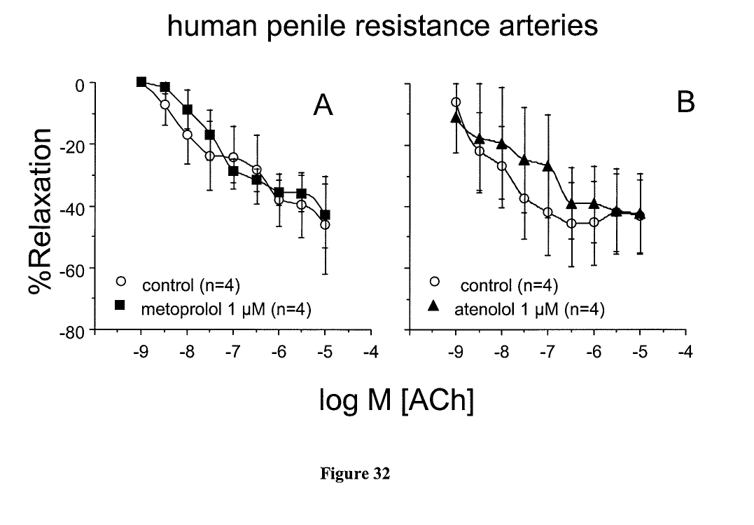

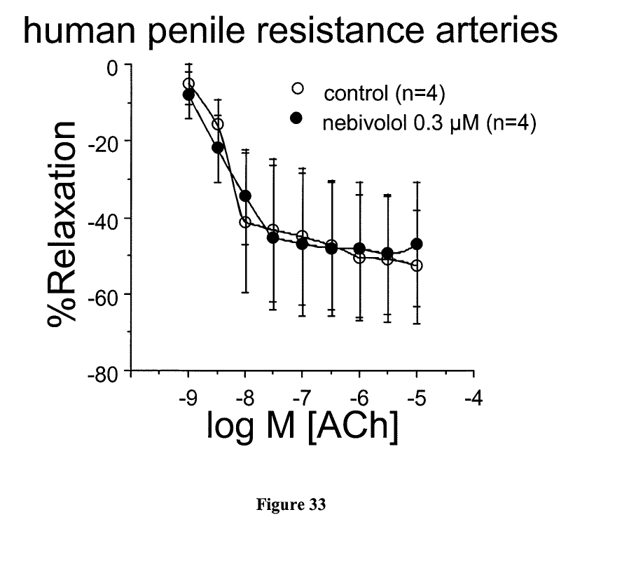

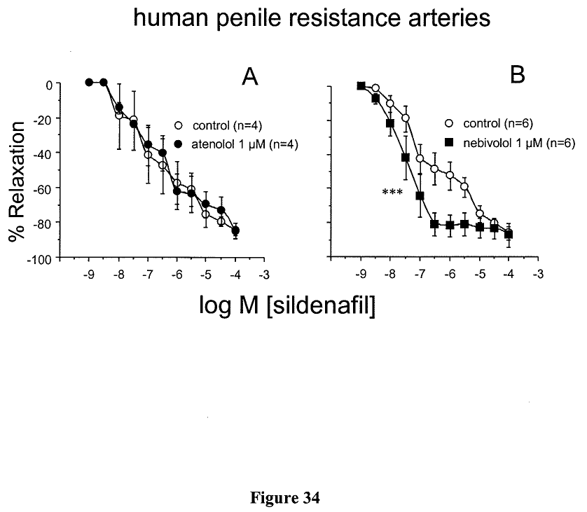

In human penile resistance arteries (HPRA) contracted with U46619 (30-100 nM), the cumulative addition of nebivolol (1 nM to 0.1 mM) produced marked relaxations of HPRA (See FIG. 30). These relaxations were significantly more potent than those caused by metoprolol or atenolol (See FIG. 31). In HPRA, endothelium-dependent relaxation caused by acetylcholine was not affected by the treatment with metoprolol (1 mM), atenolol (1 mM) or nebivolol (0.3 mM) (See FIGS. 32 and 33). However, nebivolol (1 mM), but not atenolol (1 mM), was able to enhance sildenafil-mediated relaxation of HPRA (See FIG. 34).

As can be seen from the results presented in Examples 1-5, nebivolol positively modulates NO/cGMP pathway. In vivo, nebivolol increases NOx concentration, which is prevented by NO synthase inhibition, thereby augmenting NO production. This is reinforced by the marked increase of cGMP caused by nebivolol. This cGMP increase is prevented by inhibiting NO synthase or guanylyl cyclase (the target of NO for generating cGMP) but unaffected by previous unspecific blockade of β-adrenergic receptors (AR), suggesting that the effects of nebivolol on NO/cGMP pathway are not dependent on activation of other β-AR. The activation of NO/cGMP pathway by nebivolol is not a class-related effect, since atenolol failed to produce similar effects. The activation of NO/cGMP by nebivolol is also consistent with the enhancement of PDE-5 inhibitor-induced cGMP accumulation and the potentiation of the relaxation of HCC caused by PDE-5 inhibition. At low concentrations, nebivolol is not able to significantly enhance endothelium-dependent relaxation of HCC or HPRA, but evokes a vasodilation of HPRA more efficiently than metoprolol or atenolol.

### Example 6

### Evaluation of the Effects of Nebivolol and Atenolol on Erectile Responses in Diabetic Rats in the Presence and Absence of a PDE-5 Inhibitor

### Methods

Male Sprague-Dawley rats were used as a model for this study. Diabetes was induced by a single injection of streptozotocin (STZ, 60 mg/kg; i.p.) dissolved in 0.1 M citric acid/trisodic citrate buffer (pH 4-4.5) and was assessed by glycemia determination 72 h after induction. Animals were kept, with free access to food and water, for 8 weeks before experimental assays. Eight weeks after induction of diabetes, rats show reduced erectile responses (Angulo et al., Naunyn-Schmiedeberg's Arch Pharmacol, 358(5), 529-37, 1998; Angulo et al., J. Sex Med., 2, 341-346, 2005). Blood glucose concentration was determined each week and just before the experiment. Insulin levels were not determined.

### Erectile Responses to Cavernosal Nerve Electrical Stimulation (CNES)

Rats were anesthetized with ketolar and diazepam. The surgical procedure consisted of dissection and isolation of the right cavernous nerve through an abdominal midline incision and exposure of penile crura through a transverse perineal incision. Intracavernosal pressure (ICP) measurements were permitted by insertion of a 23-gauge needle into the right crus. The right carotid artery and left external jugular vein were catheterized for constant blood pressure measurement and saline infusion, respectively. Electrostimulation was performed with a delicate platinum bipolar hook electrode connected to a stimulator and current amplifier. The parameters of electrical stimulation consisted of pulses with a duration of 0.3 ms and 1.5 mA of current intensity for 1 min. Frequency-response curves were performed by applying stimulation at 1, 3 and 10 Hz with a lapse of 3 min between each increase of frequency (Angulo et al., J. Sex Med., 2, 341-346, 2005).

### Treatment Protocol

Eight weeks after untreated diabetes, subcutaneous osmotic minipumps (Alzet, Cupertino, Calif., 2002 model) containing vehicle (50% DMSO/50% PEG 300), atenolol or nebivolol were implanted in the rats. The infusion rate was adjusted to deliver 3 mg/kg/day or 10 mg/kg/day of atenolol or nebivolol for 10 days. After this period of treatment, the diabetic rats were anesthetized and ICP responses to electrical stimulation were evaluated. After an equilibration period, the PDE5 inhibitor sildenafil (0.3 mg/kg) or the vehicle (25% glycofurol) was intravenously administered. Forty five minutes later, ICP responses were again determined

### Results

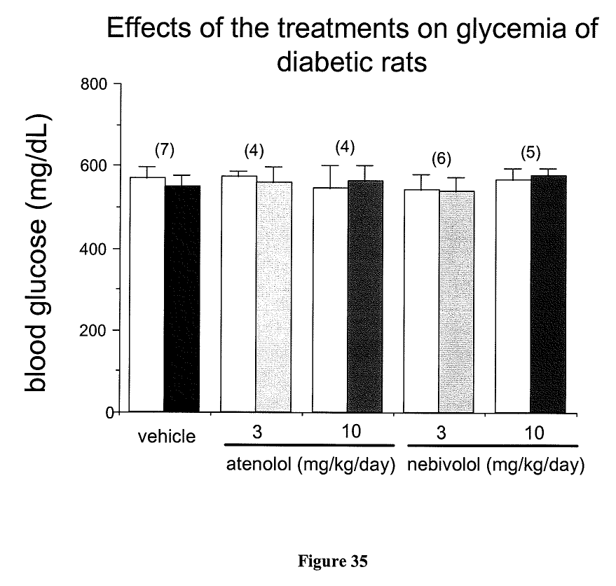

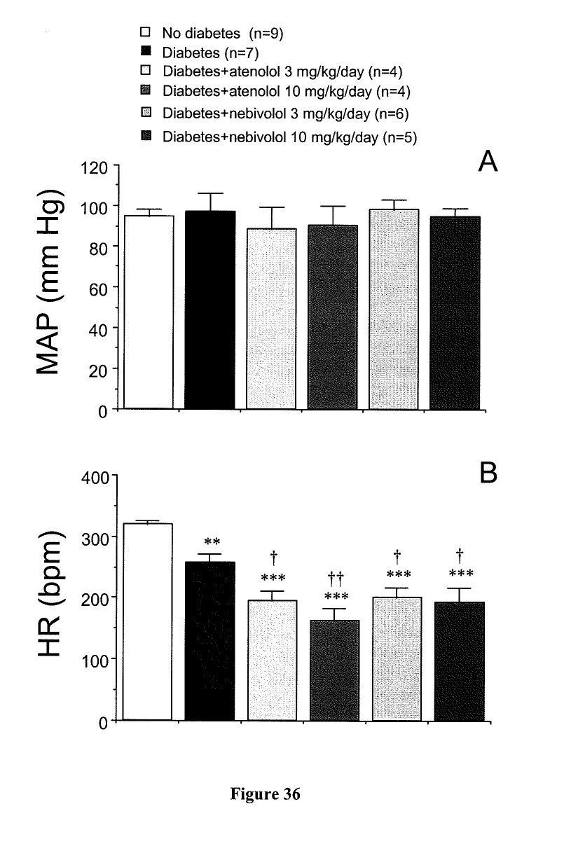

Treatment with nebivolol or atenolol (3 or 10 mg/kg/day) for 10 days did not significantly alter blood glucose concentrations in diabetic rats (See FIG. 35). Neither diabetes nor either drug treatment (nebivolol or atenolol) significantly affected arterial pressure (See FIG. 36A). The diabetic rats showed significantly reduced heart rate with respect to non diabetic rats. The heart rate was further reduced after administration of atenolol or nebivolol. (See FIG. 36B).

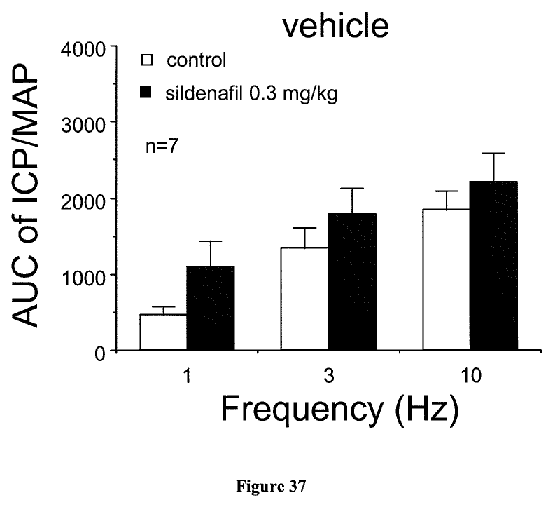

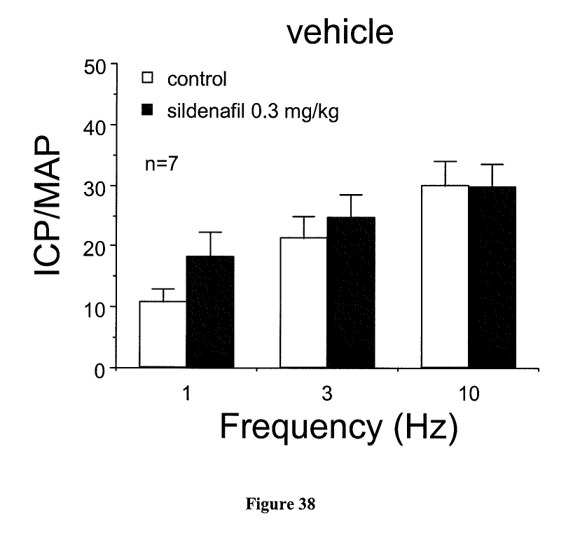

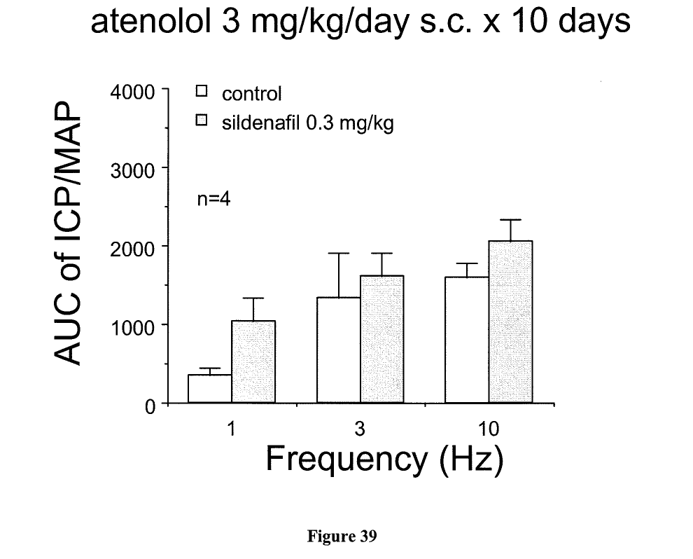

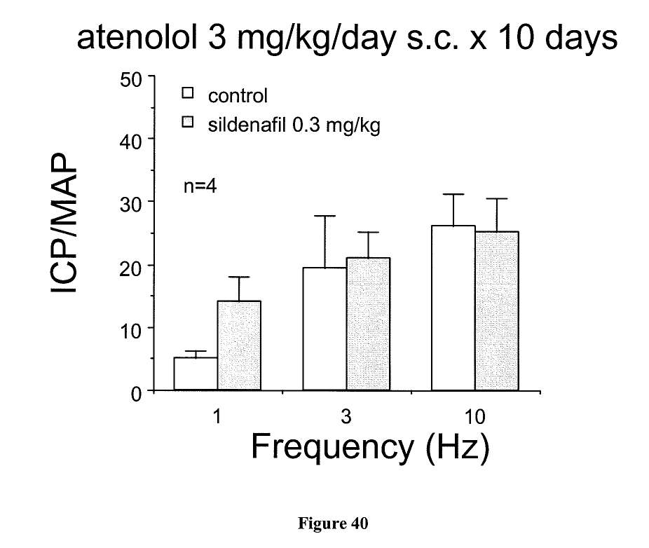

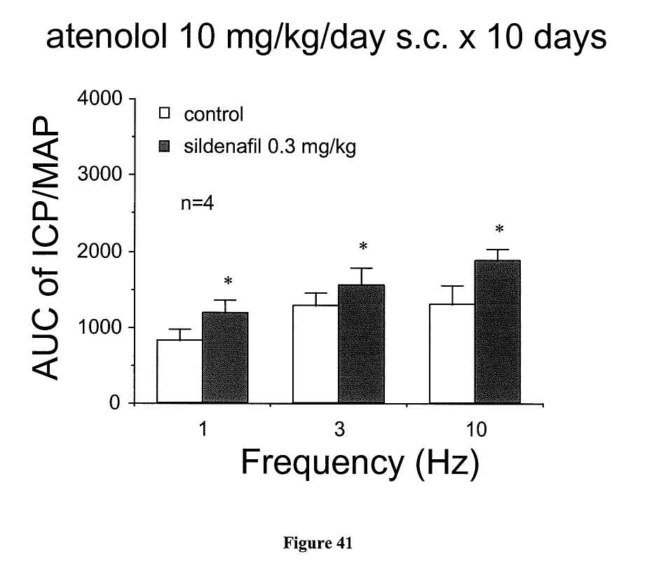

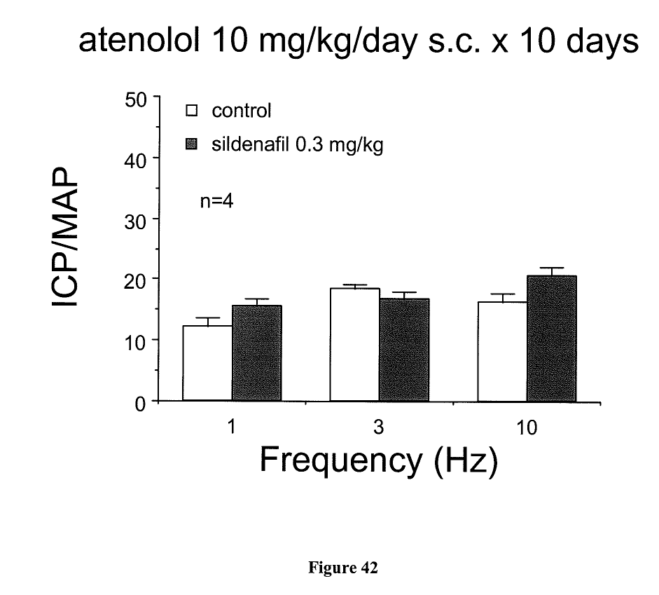

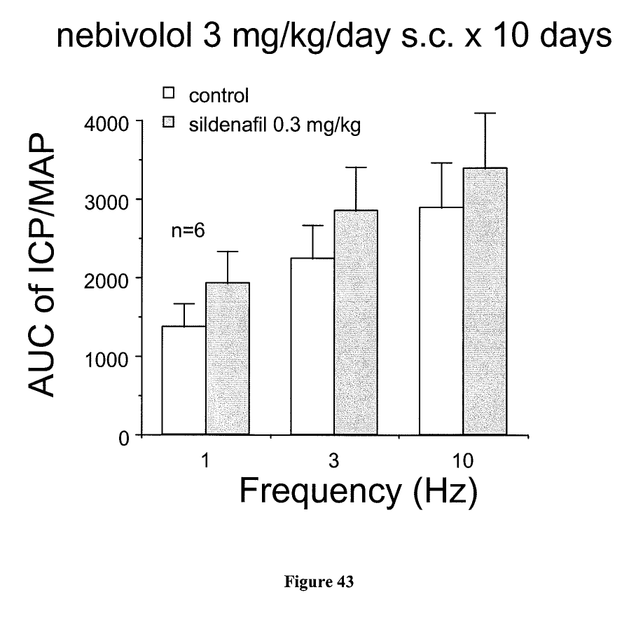

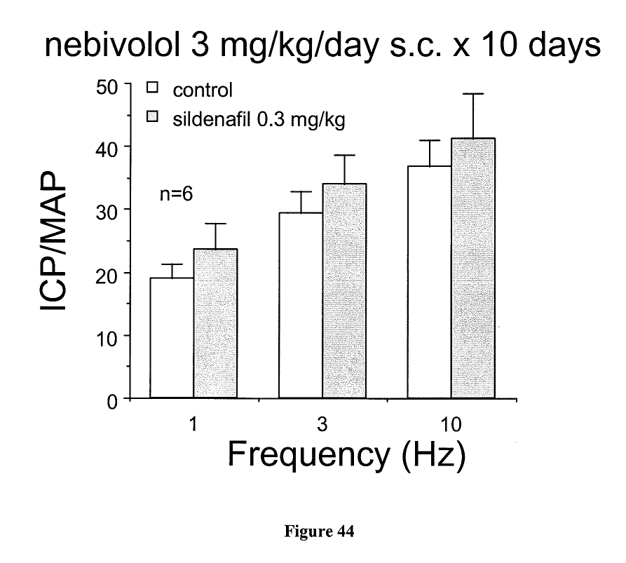

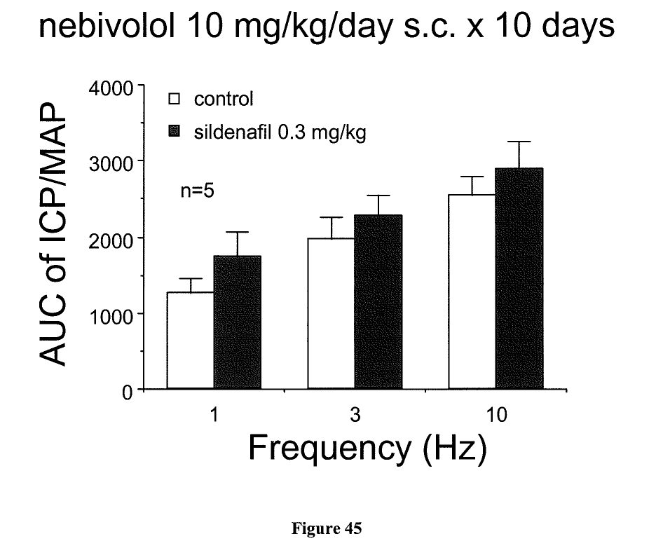

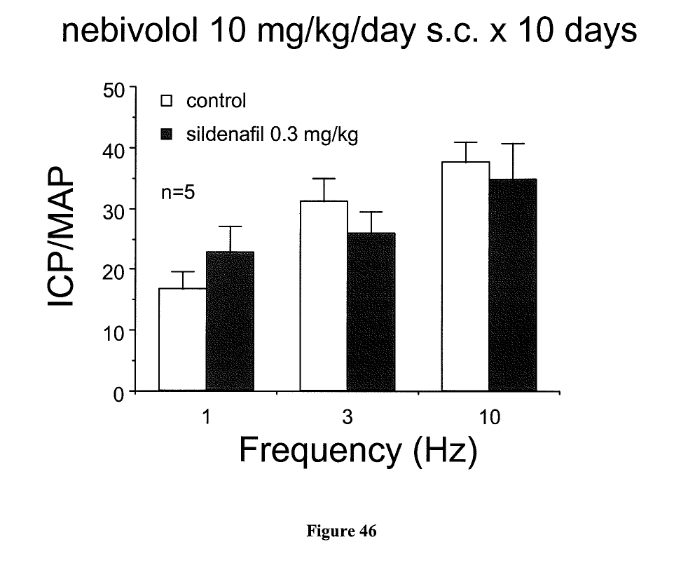

In diabetic rats, the intravenous administration of sildenafil (0.3 mg/kg; n=7) caused a potentiation of erectile responses to cavernosal nerve electrical stimulation (CNES) (See FIGS. 37 and 38). The potentiation caused by sildenafil on erectile responses did not reach statistical significance after treatment with atenolol at 3 mg/kg/day (See FIGS. 39 and 40) but in rats treated with atenolol at 10 mg/kg/day, although the potentiation was slight, it reached statistical significance when responses were measured as the AUC of ICP (See FIGS. 41 and 42). No significant potentiation by sildenafil was observed after treatment with nebivolol at a dose of 3 or 10 mg/kg/day (See FIGS. 43-46).

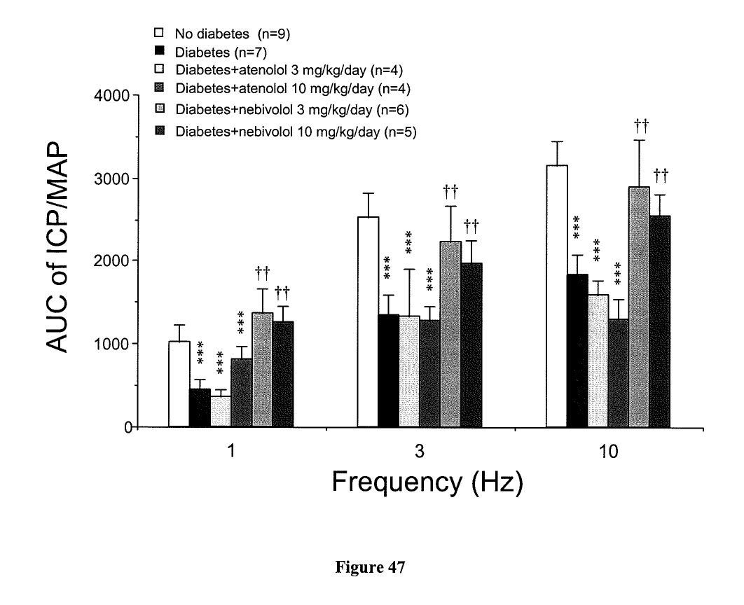

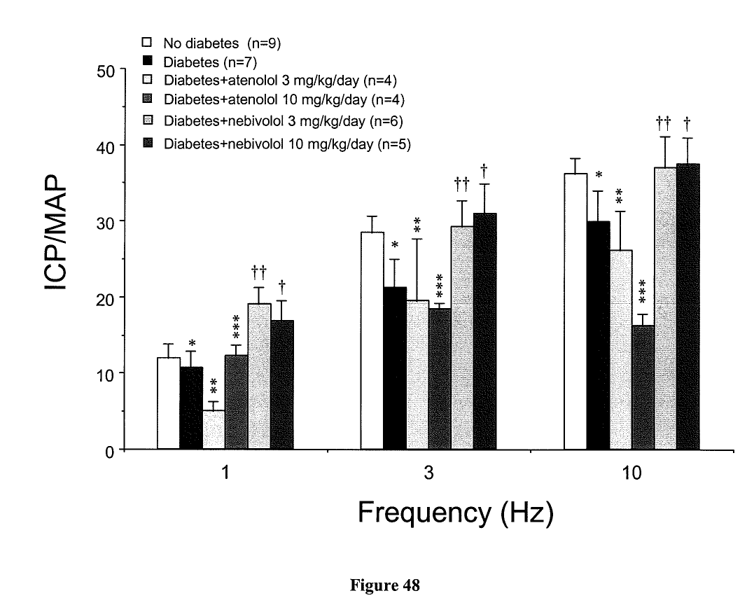

In contrast, chronic treatment for 10 days with nebivolol at both doses (3 and 10 mg/kg/day), but not atenolol (at any dose), significantly enhanced erectile responses in diabetic rats in the absence of the PDE5 inhibitor. Indeed, erectile responses in diabetic rats treated with nebivolol displayed values not significantly different from those achieved by non diabetic rats (See FIGS. 47 and 48).

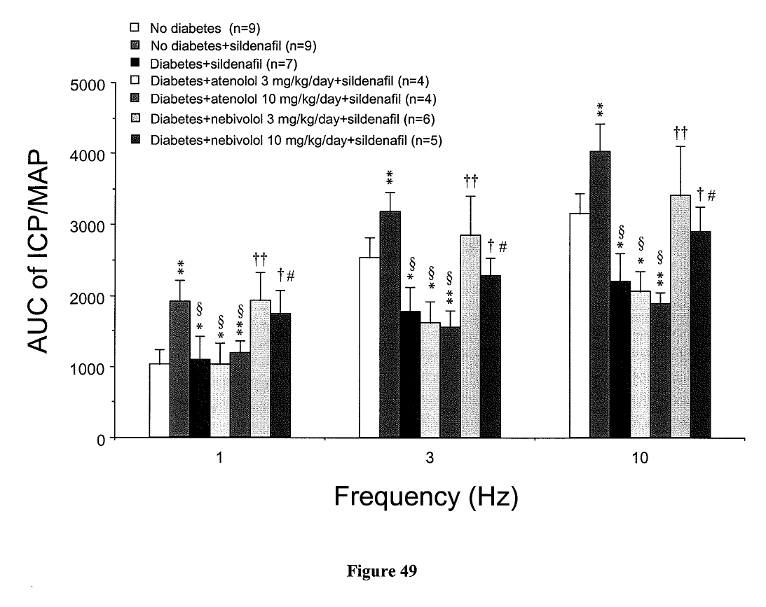

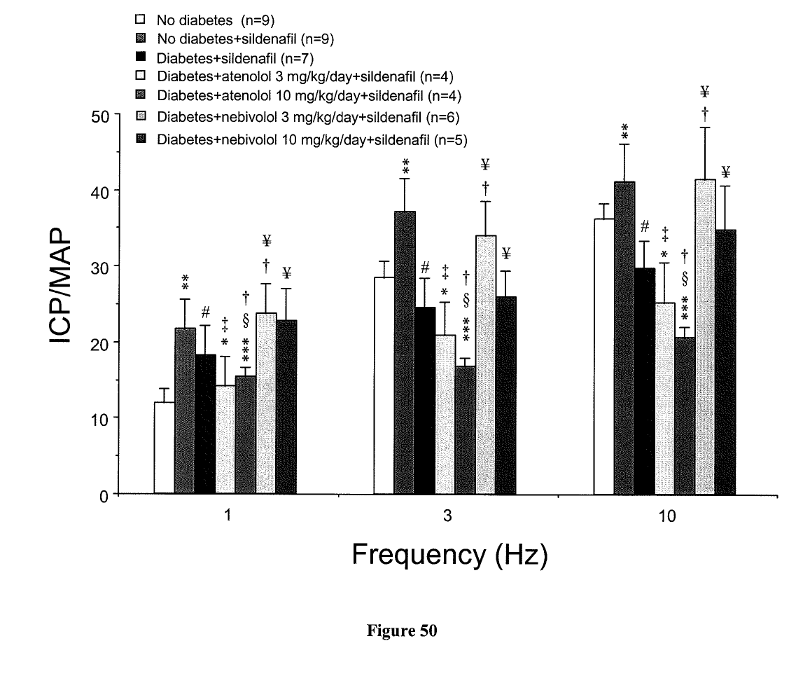

In the presence of the PDE-5 inhibitor sildenafil, rats treated with vehicle or atenolol showed significantly reduced erectile responses when compared to non diabetic rats treated with sildenafil. These reduced erectile responses were also significantly lower than that reached in non diabetic rats before administering sildenafil. In contrast, after sildenafil administration, there were not significant differences between the responses in non diabetic rats and those in nebivolol-treated diabetic rats (See FIGS. 49 and 50). There were not significant differences in the erectile responses obtained after sildenafil administration in non diabetic and in 3 mg/kg/day nebivolol-treated diabetic rats.

Increasing the dose of atenolol to 10 mg/kg/day did not positively affect erectile function in diabetic rats. In fact, erectile responses after administering the higher dose of atenolol showed a trend to be reduced with respect to vehicle-treated diabetic rats, this effect being significant after treatment with sildenafil (See FIG. 50). The 3 mg/kg/day dose of nebivolol was surprisingly equally as effective as the 10 mg/kg/day dose of nebivolol (See FIGS. 47-50).

As can be seen from the results presented in Example 6, chronic administration of nebivolol or atenolol did not affect glycemia in STZ-induced diabetic rats. Further, bradycardic effects of nebivolol and atenolol were observed in diabetic animals despite their reduced heart rates. Nebivolol administration for 10 days (3 and 10 mg/kg/day) resulted in improved erectile function in diabetic rats, an effect not observed with the β-blocker atenolol. Although sildenafil does not significantly potentiate erectile responses in diabetic rats treated with nebivolol, erectile function in these rats treated with nebivolol and sildenafil is comparable to that of non-diabetic rats and is enhanced when compared with diabetic rats only treated with sildenafil. The 3 mg/kg/day dose of nebivolol surprisingly shows a favorable profile with respect to a higher 10 mg/kg/day dose.

### Example 7

### Evaluation of the Effects of Nebivolol and Atenolol on Levels of NO-Derivatives, cGMP and Oxidative Stress Markers in Diabetic Rats in the Presence and Absence of a PDE-5 Inhibitor

### Methods

Male Sprague-Dawley rats were used as a model for this study. Diabetes was induced by a single injection of streptozotocin (STZ, 60 mg/kg; i.p.) dissolved in 0.1 M citric acid/trisodic citrate buffer (pH 4-4.5) and was assessed by glycemia determination 72 h after induction. Animals were kept, with free access to food and water, for 8 weeks before experimental assays. Eight weeks after induction of diabetes, rats show altered in vivo vascular function (Angulo et al., Naunyn-Schmiedeberg's Arch Pharmacol, 358(5), 529-37, 1998) and reduced erectile responses (Angulo et al., J. Sex Med., 2, 341-346, 2005). Blood glucose concentration was determined each week and just before the experiment. Insulin levels were not determined.

### Determination of NO Derivatives in Rat Serum.

Total NO derivatives (nitrites plus nitrates) were measured in pre-filtered (10,000 MW pore size) serum samples from the studied rats by the Griess colorimetric method, using a commercial kit for nitrite and nitrate determination from Cayman Chemical Co. (Ann Arbor, Mich.). See Granger et al., Methods Enzymol., 268, 142-151, 1996.

### Determination of TBARS in Rat Serum

Blood was collected from the right ventricle, clotted in gel and centrifuged at 1500×g to obtain serum. Thiobarbituric acid reactive substances (TBARS) were determined by reacting thiobarbituric acid with serum samples at 70° C. for 30 min and reading absorbance at 532 nm. Serum concentrations of TBARS were determined by using malondialdehyde (MDA) as a standard. MDA was obtained by hydrolysing 1,1,3,3-tetraethoxypropane with 0.1 N HCl at 100° C. for 5 min. Results were expressed as ng/ml of MDA equivalents.

Determination of cGMP Content in Rat Plasma and Penile Tissue

Plasma samples were collected, immediately frozen and stored at −80° C. until extraction for cyclic nucleotide assay. Samples were then precipitated in ethanol, centrifuged and the supernatant was dried under a nitrogen stream. Penile tissues were rapidly immersed in liquid nitrogen and were stored at −80° C. until extraction for cyclic nucleotide assay. The tissues were then extracted by homogenization in 6% trichloroacetic acid, followed by ether (H₂O-saturated) extraction and lyophilization. cGMP was determined by ELISA using a kit from Cayman Chemical Co.

### Experimental Procedures

Eight weeks after untreated diabetes, subcutaneous osmotic minipumps (Alzet, Cupertino, Calif., 2002 model) containing vehicle (PEG300:DMSO, 1:1), atenolol or nebivolol were implanted. Concentrations of the infused solutions and the infusion rate were adjusted to deliver atenolol (3 or 10 mg/kg/day) or nebivolol (3 or 10 mg/kg/day) for 10 days. After 10 days treatment, diabetic rats were anesthetized and erectile responses to cavernosal nerve electrical stimulation (CNES) were determined. After an equilibration period, the PDE-5 inhibitor sildenafil (0.3 mg/kg) or the vehicle (25% glycofurol) was intravenously administered. Forty five minutes later, erectile responses to CNES were again evaluated, and 5 min after CNES application, blood was collected for obtaining plasma and serum, and the penis was excised. Serum samples were immediately frozen until determinations of NOx and TBARS. Plasma and penile tissues were immediately frozen until determinations of cGMP.

Plasma and tissue samples for this study were obtained from the animals described in Example 6.

### Results

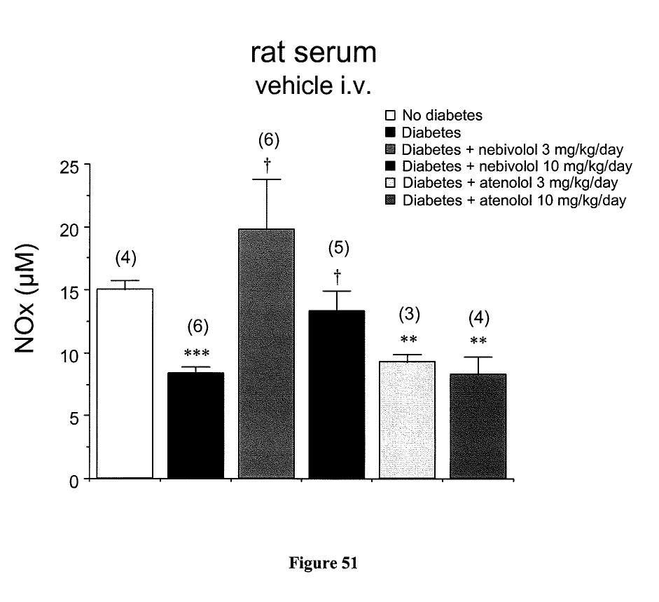

Nitrite plus nitrate (NOx) content was significantly reduced in sera from diabetic rats. Administration of nebivolol for 10 days (3 and 10 mg/kg/day s.c.) significantly increased NOx content in sera from diabetic rats, completely recovering NOx concentration up to levels not different from non-diabetic rats. This effect was surprisingly more marked with the 3 mg/kg/day dose of nebivolol (See FIG. 51).

Administration of the β-blocker atenolol for 10 days (3 and 10 mg/kg/day s.c.) did not affect NOx content in sera from diabetic rats. NOx concentration remained significantly reduced in diabetic rats treated with atenolol when compared to serum levels in non-diabetic rats (See FIG. 51).

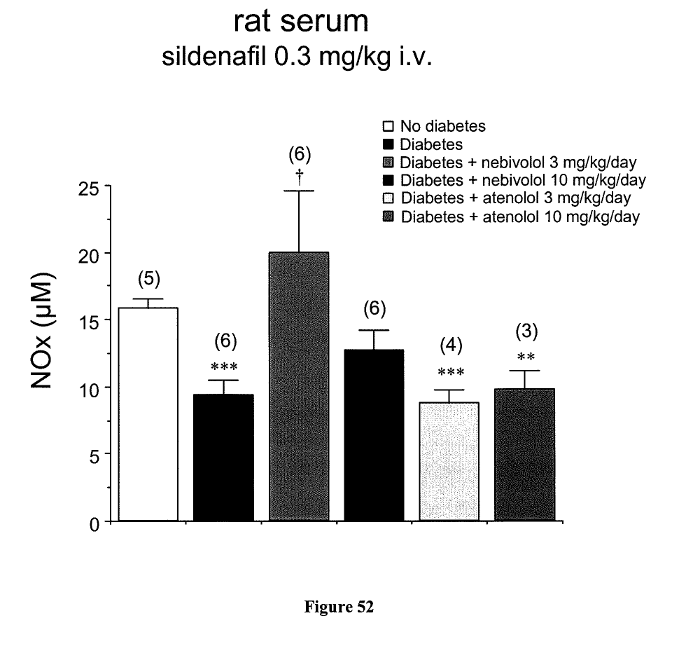

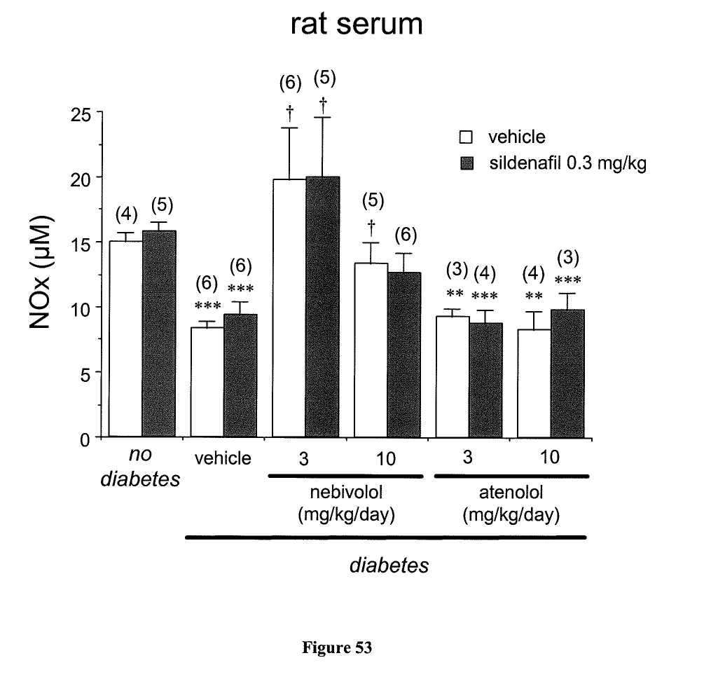

Intravenous administration of the PDE-5 inhibitor sildenafil (0.3 mg/kg) did not modify serum NOx content in rats. The effects of nebivolol and atenolol on NOx content in sera from diabetic rats were not influenced by the treatment with sildenafil (See FIGS. 52 and 53).

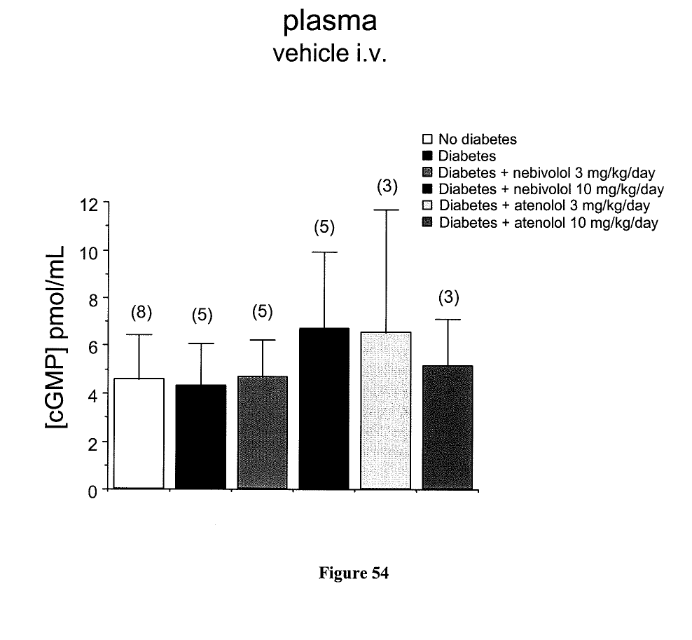

Diabetic rats did not show significant alterations in plasma cGMP content. Administration of atenolol or nebivolol, for 10 days (both at 3 and 10 mg/kg/day) did not result in significant modifications of the plasma cGMP content in diabetic rats (See FIG. 54).

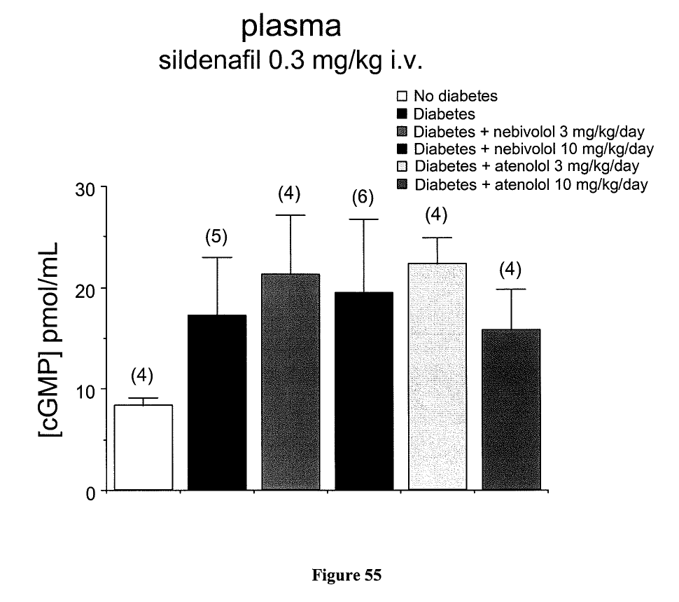

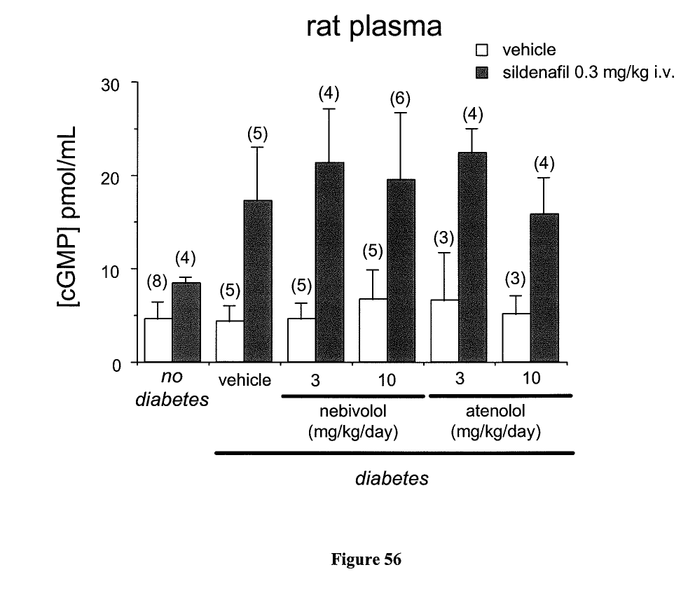

Intravenous administration of sildenafil (0.3 mg/kg) caused a marked increase in plasma cGMP levels in diabetic and non diabetic rats. After sildenafil administration, cGMP levels in plasma from diabetic rats were not lower than those achieved in non diabetic rats. Administration of atenolol or nebivolol, for 10 days (both at 3 and 10 mg/kg/day) did not influence the effects of sildenafil on plasma cGMP content in diabetic rats (See FIGS. 55 and 56).

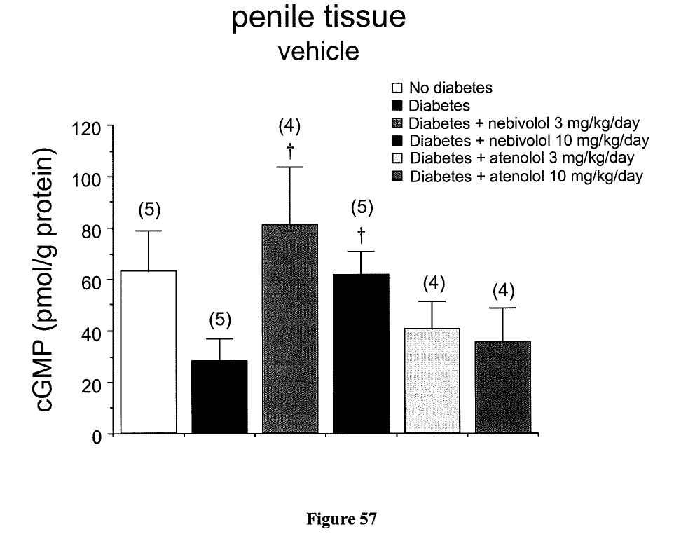

The cGMP content was numerically but not statistically significantly less in penile tissue from diabetic rats (p<0.1). However, administration of nebivolol for 10 days (3 and 10 mg/kg/day) resulted in a statistically significant increase in cGMP content in penile tissue from diabetic rats (See FIG. 57). This effect was not shown by atenolol (3 and 10 mg/kg/day).

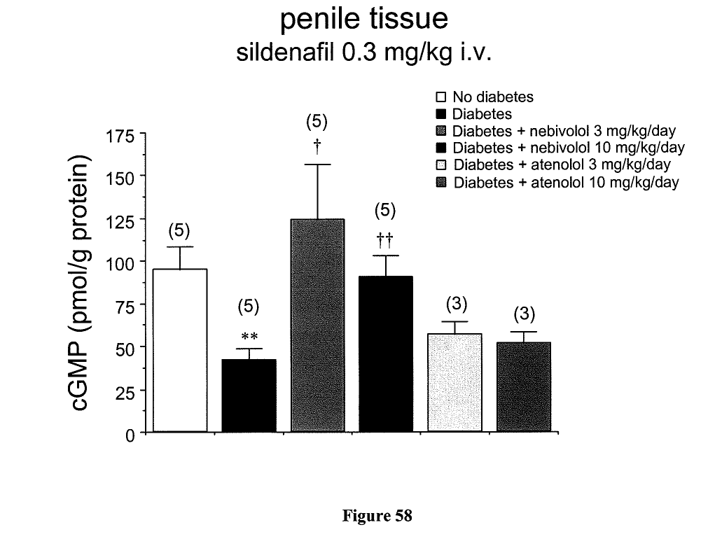

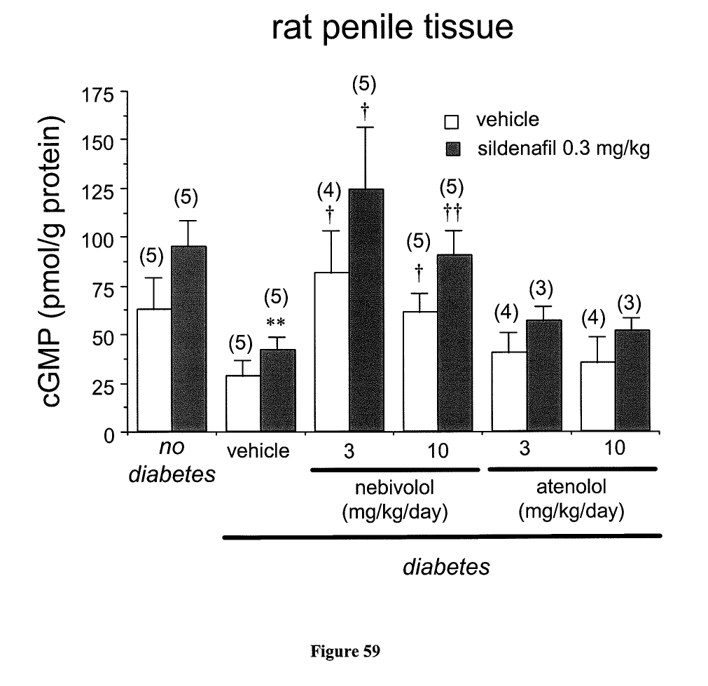

Intravenous administration of sildenafil (0.3 mg/kg) produced an increase in cGMP levels in penile tissue from diabetic and non diabetic rats. After sildenafil administration, cGMP levels in penile tissue from diabetic rats were significantly less when compared to non diabetic rats (See FIGS. 58 and 59). The penile tissue from diabetic rats treated with nebivolol for 10 days (3 and 10 mg/kg/day) after exposure to sildenafil (0.3 mg/kg) achieved cGMP levels which were not significantly different from those in non diabetic rats but significantly higher than those obtained in diabetic rats treated with vehicle for 10 days (See FIGS. 58 and 59). Treatment with atenolol (3 and 10 mg/kg/day) for 10 days did not modify the cGMP content in penile tissue from diabetic rats after administration of sildenafil (0.3 mg/kg, i.v.) (See FIGS. 58 and 59).

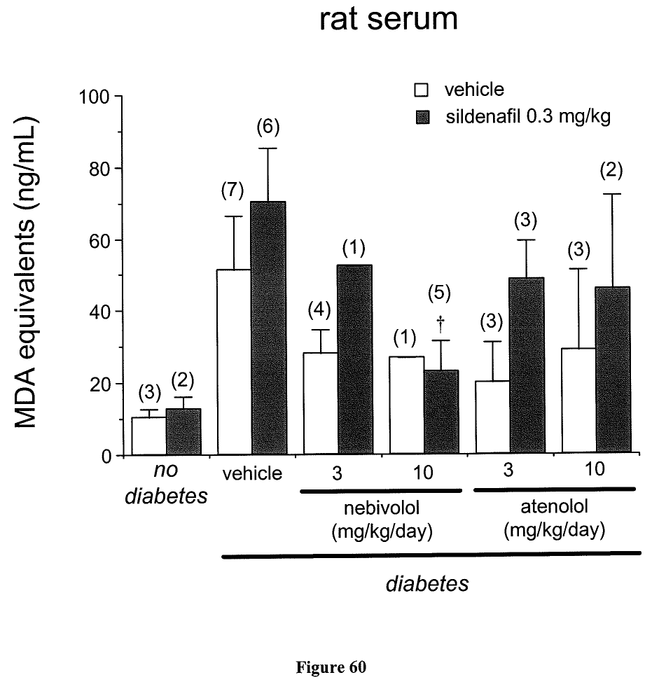

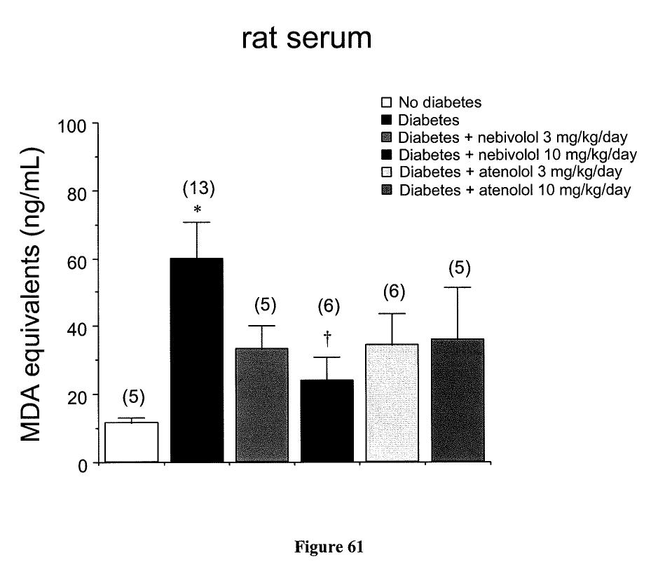

Diabetes caused a significant elevation of serum concentrations of thiobarbituric acid reactive substances (TBARS) in rats (See FIGS. 60 and 61). Sildenafil (0.3 mg/kg; i.v.) did not significantly influence TBARS levels (See FIG. 59). The results from rats of each treatment group, receiving or not receiving sildenafil, were pooled for obtaining larger number of animals for statistical comparisons. Under these conditions, both atenolol and nebivolol showed a trend to reduce TBARS levels in diabetic rats when administered for 10 days, but a significant reduction of TBARS was observed after treatment with the highest concentration of nebivolol (10 mg/kg/day) (See FIG. 61).

As can be seen from results described in Example 7, nebivolol augments serum concentrations of NO derivatives (NOx) and penile tissue content of cGMP in diabetic rats. This increase leads to the recovery of serum NOx and penile cGMP levels to those observed in non diabetic rats. A nebivolol dose of 3 mg/kg/day surprisingly appears to be more effective than a nebivolol dose of 10 mg/kg/day. The increase in cGMP levels in penile tissue observed with nebivolol was also observed after acutely treating the rats with the PDE-5 inhibitor sildenafil. In contrast, atenolol failed to modify serum NOx or penile cGMP levels in diabetic rats with or without sildenafil administration. The positive impact of nebivolol on NO/cGMP pathway in diabetes may be related in part to its capacity to reduce oxidative stress (i.e. reduction in TBARS). Atenolol showed reductions in this oxidative stress marker while not affecting the NO/cGMP pathway or erectile function.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, patent applications and publications cited throughout this application are incorporated herein by reference in their entireties.

1. A method of treating sexual dysfunction in a patient in need thereof comprising administering to the patient a therapeutically effective amount of nebivolol, or a pharmaceutically acceptable salt thereof.

2. The method of claim 1, wherein the sexual dysfunction is erectile dysfunction.

3. The method of claim 1, wherein the sexual dysfunction is female sexual arousal disorder.

4. The method of claim 1, wherein the therapeutically effective amount comprises a daily dose ranging from about 0.1 to about 20 mg per day.

5. The method of claim 1, wherein the therapeutically effective amount comprises a daily dose ranging from about 1 to about 15 mg per day.

6. The method of claim 1, wherein the therapeutically effective amount comprises a daily dose ranging from about 2.5 to about 10 mg per day.

7. The method of claim 5, wherein the therapeutically effective amount comprises a daily dose of about 2.5 mg per day.

8. The method of claim 5, wherein the therapeutically effective amount comprises a daily dose of about 5 mg per day.

9. The method of claim 5, wherein the therapeutically effective amount comprises a daily dose of about 10 mg per day.

10. The method of claim 1, wherein the therapeutically effective amount is administered in a single administration.

11. The method of claim 1, wherein the therapeutically effective amount is administered in multiple administrations.

12. The method of claim 1, wherein the nebivolol, or a pharmaceutically acceptable salt thereof, is administered orally, intravenously, sublingually, or buccally.

13. The method of claim 12, wherein the nebivolol, or pharmaceutically acceptable salt thereof, is administered orally.

14. The method of claim 1 comprising administering to the patient a therapeutically effective amount of nebivolol hydrochloride.

15. A method of treating sexual dysfunction in a patient comprising administering to the patient a therapeutically effective amount of nebivolol, or a pharmaceutically acceptable salt thereof, in combination with a second active agent.

16. The method of claim 15, wherein the sexual dysfunction is an erectile dysfunction.

17. The method of claim 15, wherein the sexual dysfunction is a female sexual arousal disorder.

18. The method of claim 15, wherein the second active agent is a type V phosphodiesterase (PDE-5) inhibitor.

19. The method of claim 18, wherein the type V phosphodiesterase inhibitor is selected from the group consisting of sildenafil, tadalafil, vardenafil, zaprinast and pharmaceutically acceptable salts thereof.

20. The method of claim 18, wherein the type V phosphodiesterase inhibitor is sildenafil, or a pharmaceutically acceptable salt thereof.

21. The method of claim 20, wherein the type V phosphodiesterase inhibitor is sildenafil citrate.

22. The method of claim 21, comprising administering a therapeutically effective amount of nebivolol hydrochloride in combination with sildenafil citrate.

23. The method of claim 18, wherein the type V phosphodiesterase inhibitor is tadalafil, or a pharmaceutically acceptable salt thereof.

24. The method of claim 18, wherein the type V phosphodiesterase inhibitor is vardenafil, or a pharmaceutically acceptable salt thereof.

25. The method of claim 18, wherein the type V phosphodiesterase 5 inhibitor is zaprinast, or a pharmaceutically acceptable salt thereof.

26. The method of claim 18, wherein the nebivolol, or pharmaceutically acceptable salt thereof, and the type 5 phosphodiesterase (PDE-5) inhibitor are administered orally, intravenously, sublingually, or buccally.

27. The method of claim 26, wherein the nebivolol, or pharmaceutically acceptable salt thereof, and the type 5 phosphodiesterase (PDE-5) inhibitor are administered orally.

28. A method of treating a sexual dysfunction in a patient in need thereof comprising administering to the patient a therapeutically acceptable amount of nebivolol, or a pharmaceutically acceptable salt thereof, and a PDE-5 inhibitor, wherein the nebivolol, or a pharmaceutically acceptable salt thereof, enhances PDE-5 inhibitor induced relaxation of human corpus cavernosum tissue by at least about 2.5%, as compared to treatment with a PDE-5 inhibitor alone.

29. The method of claim 28, wherein the PDE-5 inhibitor is sildenafil, or a pharmaceutically acceptable salt thereof.

30. A method of enhancing PDE-5 inhibitor-induced relaxation of human corpus cavernosum tissue in a patient receiving a PDE-5 inhibitor, comprising administering to the patient a therapeutically acceptable amount of nebivolol, or a pharmaceutically acceptable salt thereof, wherein PDE-5 inhibitor-induced relaxation of human corpus cavernosum tissue is enhanced by at least about 2.5%, as compared to treatment with a PDE-5 inhibitor alone.

31. The method of claim 30, wherein the PDE-5 inhibitor is sildenafil, or a pharmaceutically acceptable salt thereof.

32. A method of enhancing PDE-5 inhibitor-induced relaxation of human corpus cavernosum tissue, comprising administering to the tissue a therapeutically acceptable amount of nebivolol, or a pharmaceutically acceptable salt thereof, and a PDE-5 inhibitor, wherein the PDE-5 inhibitor-induced relaxation of human corpus cavernosum tissue is enhanced by at least about 2.5%, as compared to the relaxation level that occurs with PDE-5 inhibitor administration alone.

33. The method of claim 32, wherein the PDE-5 inhibitor is sildenafil, or a pharmaceutically acceptable salt thereof.

34. A method of treating a sexual dysfunction in a patient in need thereof comprising administering to the patient a therapeutically acceptable amount of nebivolol, or a pharmaceutically acceptable salt thereof, wherein the nebivolol, or a pharmaceutically acceptable salt thereof, relaxes contraction of the human penile resistance arteries by at least about 2.5%.

35. A method of enhancing PDE-5 inhibitor-mediated dilation of human penile resistance arteries in a patient comprising administering to the patient a therapeutically acceptable amount of nebivolol, or a pharmaceutically acceptable salt thereof, wherein dilation of the human penile resistance arteries is enhanced by at least about 2.5% as compared to the dilation level that occurs with PDE-5 inhibitor administration alone.

36. The method of claim 35, wherein the PDE-5 inhibitor is sildenafil, or a pharmaceutically acceptable salt thereof.

37. A method of treating a sexual dysfunction in a patient in need thereof comprising administering to the patient a therapeutically acceptable amount of nebivolol, or a pharmaceutically acceptable salt thereof, and a PDE-5 inhibitor, wherein the nebivolol, or a pharmaceutically acceptable salt thereof, enhances PDE-5 inhibitor induced dilation of human penile resistance arteries by at least about 2.5% as compared to the dilation level that occurs with PDE-5 inhibitor administration alone.

38. The method of claim 37, wherein the PDE-5 inhibitor is sildenafil, or a pharmaceutically acceptable salt thereof.
